SEQUENCE LISTING (3 sequences)

SEQ ID NO: 1 - DNA, 50 bp, Artificial
  nucleic acid sequence of the FL814 DNA oligomer
ccccccatattcgcgattattgcccccgcaataatcgcgaatatgggggg

SEQ ID NO: 2 - DNA, 25 bp, Artificial
  nucleic acid sequence of the FL842 DNA oligomer
ccccccatattcgcgattattgcgc

SEQ ID NO: 3 - DNA, 25 bp, Artificial
  nucleic acid sequence of the FL843 DNA oligomer
gcgcaataatcgcgaatatgggggg

(19) United States
(12) Patent Application Publication
(10) Pub. No.: US 2015/0253323 A1
(43) Pub. Date: Sep. 10, 2015

(54) METHODS AND KITS FOR HIGH THROUGHPUT SCREENING FOR COMPOUNDS TARGETING DNA-BINDING AND RNA-BINDING PROTEINS

(71) Applicants: Fenfei LENG, Palmetto Bay, FL (US); Nicole ALONSO, Miami, FL (US); Jeremy CHAMBERS, Miramar, FL (US)

(72) Inventors: Fenfei LENG, Palmetto Bay, FL (US); Nicole ALONSO, Miami, FL (US); Jeremy CHAMBERS, Miramar, FL (US)

(73) Assignee: The Florida International University Board of Trustees, Miami, FL (US)

(21) Appl. No.: 14/640,541

(22) Filed: Mar. 6, 2015

Related U.S. Application Data
(60) Provisional application No. 61/948,999, filed on Mar. 6, 2014.

(51) Int. Cl. G01N 33/543

(52) U.S. Cl. CPC G01N 33/54393

(57) ABSTRACT

The present invention provides methods of screening compounds that inhibit or enable or activate or enhance the sequence-specific or nonspecific binding of a polynucleotide binding protein to a polynucleotide fragment. Also provided are enzyme linked immunosorbent assay (ELISA) kits for screening for compounds that inhibit or enable, activate, or enhance the sequence-specific or nonspecific binding of a polynucleotide binding protein.

### CROSS-REFERENCE TO RELATED APPLICATIONS

This application claims the priority benefit of U.S. Provisional Application Ser. No. 61/948,999, filed Mar. 6, 2014, which is incorporated herein by reference in its entirety.

### GOVERNMENT SUPPORT

This invention was made with government support under grant numbers 5SC1HD063059-04 and 1R15GM109254-01A1 awarded by the National Institutes of Health. The government has certain rights in the invention.

The Sequence Listing for this application is labeled “SEQ-LIST-03-05-15-ST25.txt”, which was created on Mar. 5, 2015, and is 2 KB. The entire content is incorporated herein by reference in its entirety.

### BACKGROUND OF THE INVENTION

Protein-DNA interactions play critical roles in many essential biological events, such as DNA replication, recombination and transcription. For instance, the first step of DNA replication is the binding of the origin-binding proteins, such as DnaA for bacteria and origin recognition complex for eukaryotes, to DNA replication origins to initiate DNA replication (Erzberger, J. P. and Berger, J. M. (2006); Stillman, B. (2005); O'Donnell, M., et al. (2013)). Transcription factors, on the other hand, orchestrate specific gene expression patterns in response to developmental and/or environmental stimuli (Lobe, C. G. (1992); Osborne, C. K., et al. (2001); Benizri, E., et al. (2008)). Abnormal expression and/or aberrant regulation of certain transcription factors are involved in human oncogenesis (Darnell, J. E. Jr (2002)), and tumor proliferation and malignancy (Libermann, T. A. and Zerbini, L. F. (2006); Frank, D. A. (2013)). In fact, transcription factors are considered as important therapeutic targets due to their crucial roles in many diseases including cancers (Darnell, J. E. Jr (2002)). However, since transcription factors usually do not have enzymatic activities suitable for chemical intervention, they are considered ‘undruggable’ targets (Yan, C. and Higgins, P. J. (2013)). Nevertheless, it is possible to design chemistry to disrupt protein-DNA and/or protein-protein interactions to modulate the functionalities of transcription factors, such as c-Myc and STAT3 (signal transducer and activator of transcription 3). Indeed, several high-throughput screening methods have been used to identify inhibitors targeting protein-protein interactions (Darnell, J. E. Jr (2002); Heeres, J. T. and Hergenrother, P. J. (2011); Makley, L. N. and Gestwicki, J. E. (2013)).

PDI-ELISA is the abbreviation of the protein-DNA interaction enzyme-linked immunosorbent assay; PRI-ELISA is the abbreviation of the protein-RNA interaction enzyme-linked immunosorbent assay. PDI-ELISA was first described by Hibma and coworkers in 1994 for the study of the interactions between the active human papillomavirus type 16 (HPV 16) E2 protein and DNA as a non-radioactive alternative assay (Hibma, M. H., et al. (1994)). Since then, it has been used to study several transcriptional factors recognizing sequence-specific DNA sequences, such as p53, NFκB, AP-1, and bZIP (Jagelska, E., et al. (2002); Renard, P., et al. (2001); Kirchler, T., et al. (2010); Alonso, R., et al. (2009); Rosenau, C., et al. (2004)).

### BRIEF SUMMARY

One challenge of drug development is developing rapid and efficient high-throughput screening assays to identify inhibitors from the millions of compounds found in small molecule libraries that may target protein-DNA, protein-RNA and protein-protein interactions. The present invention provides rapid and sensitive high-throughput screening methods to survey agent libraries targeting protein-DNA and protein-RNA interactions. In some aspects of the present invention, multiwell plates can be utilized containing a particular polynucleotide of interest bound to the substrate in each well. Individual agents can be screened in each well to provide rapid and high-throughput screening.

The present invention provides kits and methods for screening candidate agents (i.e., agents), such as compounds, proteins, DNA molecules, and/or RNA molecules, that inhibit, or alternatively, enable, activate, and/or enhance the sequence-specific or nonspecific binding of a polynucleotide binding protein to a polynucleotide fragment.

In one aspect, the present invention provides methods of screening agents that inhibit the sequence-specific binding of a polynucleotide binding protein, or fragment thereof, the methods comprising: obtaining a substrate with a polynucleotide fragment bound to its surface; contacting the substrate with a candidate agent; contacting the substrate with a sequence-specific polynucleotide binding protein, wherein the sequence-specific polynucleotide binding protein is specific for at least a portion of the polynucleotide fragment bound to the substrate; and detecting whether the sequence-specific polynucleotide binding protein is bound to the polynucleotide fragment. Reduced detection of the sequence-specific polynucleotide binding protein relative to a control in the absence of the candidate agent indicates the candidate agent inhibits the sequence-specific binding of the polynucleotide binding protein, which, for example, can be from the candidate agent binding to the polynucleotide fragment and inhibiting the binding of the polynucleotide binding protein thereto.

In one embodiment, the candidate agent and the sequence-specific polynucleotide binding protein, or fragment thereof, are contacted together before contacting the substrate. Therefore, a decrease or absence of detection of the sequence-specific polynucleotide binding protein relative to control in the absence of the candidate agent signifies the candidate agent binds to the polynucleotide binding protein and prevents the sequence-specific binding of the polynucleotide binding protein to the polynucleotide fragment. In specific embodiments, the candidate agent causes a protein conformation change that prevents the sequence-specific binding of the polynucleotide binding protein to the polynucleotide fragment.

In another aspect, the present invention provides methods of screening agents that enable the sequence-specific binding of a protein, or fragment thereof, to a polynucleotide fragment, the methods comprising: obtaining a substrate with the polynucleotide fragment bound to its surface; obtaining a protein that lacks a binding affinity for the polynucleotide fragment; contacting the protein with a candidate agent to form a protein-compound mixture; contacting the substrate with the protein-compound mixture; and detecting whether the protein is bound to the polynucleotide fragment. Detection of the protein signifies the candidate agent enables the sequence-specific binding of the protein.

In another aspect, the present invention provides methods of screening agents that enable the sequence-specific binding of a protein, or fragment thereof, to a polynucleotide fragment, the methods comprising: obtaining a substrate with the polynucleotide fragment bound to its surface; contacting the substrate with a candidate agent; obtaining a protein that lacks a binding affinity for the polynucleotide fragment; contacting the substrate with the protein; and detecting whether the protein is bound to the polynucleotide fragment. Detection of the protein signifies the candidate agent is a compound that enables the sequence-specific binding of the protein.

In another aspect, the present invention provides methods of screening agents that inhibit the nonspecific binding of a polynucleotide binding protein, or fragment thereof, the methods comprising: obtaining a substrate with a polynucleotide fragment bound to its surface; contacting the substrate with a candidate agent; contacting the substrate with a nonspecific polynucleotide binding protein; and detecting whether the polynucleotide binding protein is bound to the polynucleotide fragment. Reduced detection of the nonspecific polynucleotide binding protein relative to a control in the absence of the candidate agent indicates the candidate agent inhibits the binding of the polynucleotide binding protein, which, for example, can be from the candidate agent binding to the polynucleotide fragment and inhibiting the binding of the polynucleotide binding protein thereto.

In one embodiment, the candidate agent and the nonspecific polynucleotide binding protein, or fragment thereof, are contacted together before contacting the substrate. Therefore, reduced detection (relative to the control in the absence of the candidate agent) of the nonspecific polynucleotide binding protein signifies the candidate agent binds to the polynucleotide binding protein and prevents the binding of the polynucleotide binding protein to the polynucleotide fragment. In specific embodiments, the candidate agent causes a protein conformational change that prevents the binding of the polynucleotide binding protein to the polynucleotide fragment.

In another aspect, the present invention provides methods of screening agents that inhibit or, alternatively, enable or enhance the sequence-specific binding of a polynucleotide binding protein, or fragment thereof, the methods comprising: providing a substrate with a plurality of a polynucleotide fragment bound to its surface; contacting the substrate with an amount of a candidate agent; contacting the substrate with a plurality of a sequence-specific polynucleotide binding protein, wherein the sequence-specific polynucleotide binding protein binds at least a portion of the polynucleotide fragment bound to the surface; contacting the substrate with a first antibody specific for the sequence-specific polynucleotide binding protein; contacting the substrate with a detector antibody specific for the first antibody to effect an immunoreaction, the detector antibody comprising a label that provides a detectable signal; detecting the detectable signal; and quantifying the detectable signal to determine the amount of the sequence-specific polynucleotide binding protein bound to the polynucleotide fragment. Reduced detection of the detectable signal relative to a control in the absence of the candidate agent indicates the candidate agent inhibits the sequence-specific binding of the polynucleotide binding protein; increased detection of the detectable signal relative to the control in the absence of the candidate agent indicates the candidate agent enhances or enables the sequence-specific binding of the polynucleotide binding protein. In some embodiments, reduced detection of the detectable signal relative to a control in the absence of the candidate agent indicates the candidate agent is an agent that binds to the polynucleotide fragment and inhibits the binding of the polynucleotide binding protein thereto. Furthermore, the candidate agent and the sequence-specific polynucleotide binding protein can be contacted together before contacting the substrate such that reduced detection of the detectable signal relative to a control (in the absence of the candidate agent) indicates the candidate agent binds to the polynucleotide binding protein and disrupts the sequence-specific binding of the polynucleotide binding protein to the polynucleotide fragment. In such embodiments, the candidate agent causes a protein conformation change that disrupts the sequence-specific binding of the polynucleotide binding protein to the polynucleotide fragment.

In yet another aspect, the present invention provides methods of screening for agents that enable the sequence-specific binding of a protein, or fragment thereof, to a polynucleotide fragment, the methods comprising: providing a substrate with a plurality of a polynucleotide fragment bound to its surface; contacting the substrate with an amount of a candidate agent; contacting the substrate with a protein that lacks a binding affinity for the polynucleotide fragment; contacting the substrate with a first antibody specific for the protein; contacting the substrate with a detector antibody specific for the first antibody to effect an immunoreaction, the detector antibody comprising a label that provides a detectable signal; detecting the detectable signal; quantifying the detectable signal to determine the amount of the protein bound to the polynucleotide fragment. Increased detection of the detectable signal relative to a control in the absence of the candidate agent indicates the candidate agent enables the sequence-specific binding of the protein. Furthermore, the candidate agent and the protein can be contacted together before contacting the substrate such that increased detection of the detectable signal relative to a control (in the absence of the candidate agent) indicates the candidate agent binds to the protein and enables the sequence-specific binding of the protein to the polynucleotide fragment. In such embodiments, the candidate agent may cause a protein conformation change that enables the sequence-specific binding of the protein to the polynucleotide fragment.

In some embodiments of aspects described herein, the detectable signal is generated by enzyme-linked immunosorbent assay (ELISA).

In another aspect, the present invention provides enzyme linked immunosorbent assay (ELISA) kits for screening for agents that inhibit, or alternatively enable, the sequence-specific or nonspecific binding of a polynucleotide binding protein, or fragment thereof, the kit comprising: a substrate for binding a polynucleotide fragment of interest; a known amount of a polynucleotide binding protein, or fragment thereof; a known amount of a first antibody specific to the polynucleotide binding protein, or fragment thereof; and a known amount of a labelled-secondary antibody specific to the first antibody.

In some embodiments, the polynucleotide fragment of interest is bound to the substrate. In some embodiments, the substrate is a multi-well plate, such as for example, but not limited to, 6, 12, 24, 96, 384, and 1536 well plates.

In embodiments of the methods and kits of the present invention, the polynucleotide fragment is selected from the group consisting of a DNA oligomer, an RNA oligomer, double stranded DNA, double stranded RNA, single stranded DNA, and single stranded RNA.

The methods and kits herein described can be used in connection with pharmaceutical, medical, and veterinary applications, as well as fundamental scientific research and methodologies, as would be identifiable by a skilled person upon reading of the present disclosure. These and other objects, features and advantages of the present invention will become clearer when the drawings as well as the detailed description are taken into consideration.

### BRIEF DESCRIPTION OF DRAWINGS

For a fuller understanding of the nature of the present invention, reference should be had to the following detailed description taken in connection with the accompanying figures.

FIGS. 1A-1C show an illustration of binding of DNA oligomers or fragments to a solid surface: the synthetic biotin-labeled oligomers (hairpins or double stranded oligomers or single stranded oligomers) can bind to streptavidin-coated substrates (A); the synthetic oligomers can be covalently linked to pre-activated substrate surfaces (B); and the long biotin-labeled DNA fragments can be generated using PCR followed by DNA ligation and binding to streptavidin molecules on the substrate surface (C).

FIG. 2 shows an embodiment of the present invention of high throughput compound screening using PDI-ELISA such that the compound binds to the protein-binding site of the DNA fragment and inhibits the binding of the sequence-specific DNA-binding protein.

FIG. 3 shows an embodiment of the present invention of high throughput compound screening using PDI-ELISA where the compound binds to the protein and inhibits the binding of the sequence-specific DNA-binding protein to the DNA oligomer.

FIG. 4 shows an embodiment of the present invention of high throughput compound screening using PDI-ELISA where the protein does not bind to the DNA oligomer. However, a small compound molecule binds to the protein and changes the protein conformation such that the compound-protein complex specifically binds to the DNA-binding site of the oligomer.

FIG. 5 shows an embodiment of the present invention of high throughput compound screening using PDI-ELISA where the compound binds to the DNA-binding protein and causes the protein conformation change. In this way, the compound-protein complex is not able to bind to the DNA oligomer.

FIG. 6 shows an embodiment of the present invention of high throughput compound screening using PDI-ELISA where the compound binds to DNA and causes the DNA conformation change. This conformation changes enable or stimulate the protein binding to DNA.

FIGS. 7A-7C show an illustration of (A) the recognition of DNA minor groove by the “AT hook” DNA binding domain of HMGA proteins; (B) the biotin-labeled DNA oligomer FL814; and (C) the chemical structure of netropsin.

FIG. 8 shows a high throughput procedure for screening compounds against HMGA2-DNA interactions utilizing netropsin as proof of concept.

FIG. 9 shows titration experiments of HMGA2 binding to the oligomer FL814 carrying the SELEX 1 in 2×SSCT. Different dilutions of the second antibody, i.e., Anti-rabbit IgG, HRP-linked antibody, were used in these experiments.

FIGS. 10A-10B show netropsin completely inhibited HMGA2 binding to FL814. (A) Two dilutions of the second antibody i.e., Anti-rabbit IgG, HRP-linked Antibody, were used in these experiments. (B) The netropsin titration experiments were used to determine the apparent inhibitory IC₅₀.

FIGS. 11A-11B show inhibition of HMGA2 binding to FL814 by netropsin. (A) The titration experiment of HMGA2 to the DNA substrate FL814 in 2×SSCT. The DNA dissociation constant was determined to be 0.86±0.12 μM according to the method described in Materials and Methods. The standard deviation was calculated according to three different titration experiments. (B) The netropsin titration experiments were used to determine the apparent inhibitory IC₅₀ (9.30±0.78 nM) using the following equation: % HMGA2 binding to DNA=100/[1+C*(1+Kc2*C)/[IC₅₀(1+Kc2*IC₅₀)]], where Kc2 is a macroscopic binding constant for inhibitor binding to DNA, IC₅₀ is the concentration of inhibitor that causes 50% inhibition of HMGA2 binding to DNA, and C is the concentration of inhibitor. The standard deviation was calculated according to three different titration experiments.

FIG. 12 shows the inhibition of HMGA2-FL814 interactions by various DNA-binding compounds at 100 nM in 2×SSCT according to the values in Table 1. Hst, EB, and Dox, represent netropsin, Hoechst 33258, ethidium bromide, and doxorubicin, respectively. Netropsin, WP631, DAPI, and Hoechst 33258 were able to completely inhibit the HMGA2-FL814 interactions under these experimental conditions.

FIG. 13 shows the chemical structures of meso-tetra(N-methyl-4-pyridyl) porphine tetratosylate (TMPyP4) and meso-tetra(N-methyl-2-pyridyl) porphine tetrachloride (TMPyP2).

FIG. 14 shows the enhancement of HMGA2 binding to the DNA oligomer FL814 by TMPyP4. The titration experiments using different concentrations of TMPyP4 (circles) and TMPyP2 (squares) were performed as described herein. 100 nM of FL814, 1 μM of HMGA2, and 1:5000 dilution of the second antibody, Anti-rabbit IgG were used in these experiments.

FIGS. 15A-15B show (A) a titration experiment of HMGA2 to FL814 in 2×SSCT in the presence of 100 nM of TMPyP4. The apparent DNA dissociation constant was determined to be 0.19±0.02 μM according to the method described in the Materials and Methods. (B) shows titration experiments using different concentrations of TMPyP4 (circles) as described in the Materials and Methods. The enhancement of HMGA2 binding to the annealing product of the oligomers FL842 and FL843 by TMPyP4 was observed. 100 nM of the annealing product, 1 μM of HMGA2, and 1:2500 dilution of the second antibody, Anti-rabbit IgG were used in these experiments. The DNA sequences of FL842 and FL843 are the sequences of SEQ ID NO:2 and SEQ ID NO:3, respectively.

FIGS. 16A-16B illustrate (A) a competition dialysis experiment diagram adapted from Ren, J. & Chaires, J. B. (1999); and (B) a bar graph showing results of the experiment. Two disposable dialysis units, each containing 0.5 mL of either 3 μM of FL814 or 3 μM of the FL814-HMGA2 complex are dialyzed against 0.73 μM of TMPyP4 for 72 hours.

FIGS. 17A-17G show that inhibition of HMGA2 by netropsin prevents 3T3-L1 adipogenesis. (A) Western blot analysis was used to confirm the increased expression of HMGA2 during adipogenesis. FABP4 was used as a control for increased gene expression during early adipogenesis, while actin was used as a loading control. (B) The fluorescent intensity of HMGA2 expression was quantified using the Image Studio Software (LI-COR Bioscience). The expression level of HMGA2 was measured across four biological replicates. (C) Adipogenesis was quantitatively measured by Oil Red O staining of 3T3-L1 cells. This method was employed to calculate the IC₅₀ of Netropsin for this process over a dose curve. An image of this approach is displayed. (D) The absorbance of Oil Red O was determined using absorbance at 492 nm. C-75, an inhibitor of fatty acid synthase, was used as a control for inhibition of adipogenesis. (E) Oil Red O staining was also assessed qualitatively in the presence and absence of netropsin using phase microscopy. (F) The impact of netrosin exposure on HMGA2 expression levels were assessed using an in-cell western approach. TO-PRO-3 was used to normalize HMGA2 expression levels to cellular DNA content. (G) Normalized HMGA2 expression was assessed in 3T3-L1 cells exposed to DMSO and netropsin.

FIGS. 18A-18C illustrate various schemes to screen inhibitors or activators targeting protein-DNA interactions by PDI-ELISA. (A) This screening scheme can be used to identify small molecule inhibitors that upon bind to the DNA-binding proteins cause the protein conformation change and dissociate the protein from the target DNA sequence. (B) Certain transcription factors do not bind to the target DNA sequence in the absence of a small molecule ligand. This screening scheme can be used to identify these small molecule ligands. (C) Certain small molecules or proteins act as activators to help a protein binding to a target DNA sequence. This screening scheme can be used to identify such activators using PDI-ELISA. Refer to FIG. 2 for the meaning of different symbols.

### BRIEF DESCRIPTION OF THE SEQUENCES

SEQ ID NO:1 is a nucleic acid sequence of the FL814 DNA oligomer.
SEQ NO:2 is a nucleic acid sequence of the FL842 DNA oligomer,
SEQ ID NO:3 is a nucleic acid sequence of the FL843 DNA oligomer.

### DETAILED DISCLOSURE

Several aspects of the invention are described below, with reference to examples for illustrative purposes only. It should be understood that numerous specific details, relationships, and methods are set forth to provide a full understanding of the invention. One having ordinary skill in the relevant art, however, will readily recognize that the invention can be practiced without one or more of the specific details or practiced with other methods, protocols, reagents, cell lines and animals. The present invention is not limited by the illustrated ordering of acts or events, as some acts may occur in different orders and/or concurrently with other acts or events. Furthermore, not all illustrated acts, steps or events are required to implement a methodology in accordance with the present invention. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which the invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

Reference is made to particular features (including method steps) of the invention. The disclosure of the invention in this specification includes all possible combinations of such particular features. For example, where a particular feature is disclosed in the context of a particular aspect or embodiment of the invention, that feature can also be used, to the extent possible, in combination with and/or in the context of other particular aspects and embodiments of the invention, and in the invention generally.

The term “comprises” is used herein to mean that other elements, steps, etc. are optionally present. When reference is made herein to a method comprising two or more defined steps, the steps can be carried in any order or simultaneously (except where the context excludes that possibility), and the method can include one or more steps which are carried out before any of the defined steps, between two of the defined steps, or after all of the defined steps (except where the context excludes that possibility).

The present invention provides methods and related kits utilizing enzyme-linked immunosorbent assay (ELISA) for screening agents that inhibit, or alternatively, enable, activate, or enhance the sequence-specific binding of a polynucleotide binding protein to a polynucleotide fragment. The kits and methods of the present invention advantageously provide agent screening and discovery tools for DNA- and RNA-binding proteins. The methods described herein are useful for screening candidate drugs against DNA-binding or RNA-binding proteins, including for example transcription factors, using PDI-ELISA or PRI-ELISA. PDI-ELISA is the abbreviation for protein-DNA interaction enzyme-linked immunosorbent assay; PRI-ELISA is the abbreviation of the protein-RNA interaction enzyme-linked immunosorbent assay. Furthermore, the PDI-ELISA and PRI-ELISA assays described herein can be used to screen candidate agents, such as but not limited to, compounds, proteins, DNA molecules, and RNA molecules, that target DNA- and RNA-binding proteins (or protein-DNA complexes or protein-RNA complexes), including sequence-specific or non-specific DNA- and RNA-binding proteins in a high throughput format or any other formats. The polynucleotide binding proteins that can be utilized in aspects of the methods and kits of the present invention include full-length proteins, protein fragments, and peptides.

In one aspect, the present invention provides methods of screening agents that inhibit the sequence-specific binding of a polynucleotide binding protein, the methods comprising: obtaining a substrate with a polynucleotide fragment bound to its surface; contacting the substrate surface with a candidate agent; contacting the substrate surface with a sequence-specific polynucleotide binding protein, wherein the sequence-specific polynucleotide binding protein is specific for at least a portion of the polynucleotide fragment bound to the surface; and detecting whether the sequence-specific polynucleotide binding protein is bound to the polynucleotide fragment. A lack of detection of the sequence-specific polynucleotide binding protein indicates the candidate compound is a compound that inhibits the sequence-specific binding of the polynucleotide binding protein, which, for example, can be from the candidate agent binding to the polynucleotide fragment and inhibiting the binding of the polynucleotide binding protein thereto. Detection of the sequence-specific polynucleotide binding protein indicates the candidate compound is a compound that enhances or enables the sequence-specific binding of the polynucleotide binding protein, or fragment thereof.

In some embodiments, the detecting step further comprises: contacting the substrate with a first antibody specific for the sequence-specific polynucleotide binding protein, or fragment thereof; contacting the substrate with a detector antibody specific for the first antibody to effect an immunoreaction, the detector antibody comprising a label that provides a detectable signal; detecting the detectable signal; and quantifying the detectable signal to determine the amount of the sequence-specific polynucleotide binding protein, or fragment thereof, bound to the polynucleotide fragment; wherein reduced detection of the detectable signal relative to a control in the absence of the candidate agent indicates the candidate agent inhibits the sequence-specific binding of the polynucleotide binding protein, or fragment thereof; and wherein increased detection of the detectable signal relative to the control in the absence of the candidate agent indicates the candidate agent enhances or enables the sequence-specific binding of the polynucleotide binding protein, or fragment thereof.

In one embodiment, the candidate agent and the sequence-specific polynucleotide binding protein are contacted together before contacting the substrate. Therefore, an absence or reduction of detection, relative to the control, of the sequence-specific polynucleotide binding protein signifies the candidate agent binds to the polynucleotide binding protein and prevents the sequence-specific binding of the polynucleotide binding protein to the polynucleotide fragment. In specific embodiments, the candidate agent causes a protein conformational change that prevents the sequence-specific binding of the polynucleotide binding protein to the polynucleotide fragment.

The methods described herein utilize controls as a reference in the detection steps. The reference or baseline generated by the control is determined by performing the same steps in the method without including the candidate agent. As such, the resulting detectable output of the control method is independent of any effect of the candidate agent. This generates a baseline (reference signal) for comparison to the experimental groups containing the step of introducing the candidate agent for screening.

As used herein, a “substrate” refers to any surface that provides a point of attachment for the polynucleotide fragments and enables one to contact the agents and proteins of the present invention with the fragments. Furthermore, the substrate provides a surface that can be readily washed between steps of the methods described herein. Substrates useful in the present invention include, but are not limited to, petri dishes, tissue culture dishes, multi-well plates, glass, glass beads, plastic beads including thermoplastic beads, nano particles, plastic and glass pipettes, metals, metal particles/beads, electromagnetic particles, magnetic particles/beads, organic matrices, inorganic scaffolds, and other materials that provide a solid surface

In another aspect, the present invention provides methods of screening agents that enable/activate the sequence-specific binding of a protein, or fragment thereof, to a polynucleotide fragment, the methods comprising: obtaining a substrate with the polynucleotide fragment bound to its surface; obtaining a protein, or fragment thereof, that lacks a binding affinity for the polynucleotide fragment; contacting the protein, or fragment thereof, with a candidate agent to form a protein-compound mixture; contacting the substrate surface with the protein-compound mixture; and detecting whether the protein, or fragment thereof, is bound to the polynucleotide fragment. Detection of the protein, or fragment thereof, signifies the candidate agent enables the sequence-specific binding of the protein.

In some embodiments, the detecting step further comprises: contacting the substrate with a first antibody specific for the protein, or fragment thereof; contacting the substrate with a detector antibody specific for the first antibody to effect an immunoreaction, the detector antibody comprising a label that provides a detectable signal; detecting the detectable signal; and quantifying the detectable signal to determine the amount of the protein bound to the polynucleotide fragment; wherein increased detection of the detectable signal relative to a control in the absence of the candidate agent indicates the candidate agent enables the sequence-specific binding of the protein, or fragment thereof.

In another aspect, the present invention provides methods of screening agents that enable/activate the sequence-specific binding of a protein, or fragment thereof, to a polynucleotide fragment, the methods comprising: obtaining a substrate with the polynucleotide fragment bound to its surface; contacting the substrate with a candidate agent; obtaining a protein, or fragment thereof, that lacks a binding affinity for the polynucleotide fragment; contacting the substrate surface with the protein; and detecting whether the protein, or fragment thereof, is bound to the polynucleotide fragment. Detection of the protein, or fragment thereof, signifies the candidate agent is a compound that enables the sequence-specific binding of the protein.

In some embodiments, the detecting step further comprises: contacting the substrate with a first antibody specific for the protein, or fragment thereof; contacting the substrate with a detector antibody specific for the first antibody to effect an immunoreaction, the detector antibody comprising a label that provides a detectable signal; detecting the detectable signal; and quantifying the detectable signal to determine the amount of the protein bound to the polynucleotide fragment; wherein increased detection of the detectable signal relative to a control in the absence of the candidate agent indicates the candidate agent enables the sequence-specific binding of the protein, or fragment thereof.

In another aspect, the present invention provides methods of screening agents that inhibit the nonspecific binding of a polynucleotide binding protein, or fragment thereof, the methods comprising: obtaining a substrate with a polynucleotide fragment bound to its surface; contacting the substrate surface with a candidate agent; contacting the substrate surface with a nonspecific polynucleotide binding protein, or fragment thereof; and detecting whether the polynucleotide binding protein, or fragment thereof, is bound to the polynucleotide fragment. Absence or decreased detection of the nonspecific polynucleotide binding protein relative to a control in the absence of a candidate agent signifies the candidate agent is a compound that inhibits the binding of the polynucleotide binding protein, or fragment thereof, which, for example, can be from the candidate agent binding to the polynucleotide fragment and inhibiting the binding of the polynucleotide binding protein, or fragment, thereto.

In one embodiment, the candidate agent and the nonspecific polynucleotide binding protein, or fragment thereof, are contacted together before contacting the substrate surface. Therefore, an absence or reduced detection, relative to a control in the absence of a candidate agent, of the nonspecific polynucleotide binding protein signifies the candidate agent binds to the polynucleotide binding protein and prevents the binding of the polynucleotide binding protein to the polynucleotide fragment. In specific embodiments, the candidate agent causes a protein conformation change that prevents the binding of the polynucleotide binding protein, or fragment thereof, to the polynucleotide fragment.

In the methods described herein, the detection steps can utilize labels that are attached to proteins, compounds, polynucleotides, antibodies, etc., such that colorimetric, chemiluminescence, or fluorescence methods can be used to detect whether the agents inhibit or enhance/enable the binding capacities of the nucleic acid binding proteins. Non-limiting examples of labels that can be utilized include chemiluminescent labeled antibodies such as HRP-linked antibodies and alkaline phosphatase-labeled antibodies, Bioluminescent labeled antibodies such as luciferase-conjugated antibodies, fluorescence-conjugated antibodies including near-infrared fluorescence labels, antibody-conjugated nanoparticles (magnetic nanoparticles or beads and electromagnetic nano particles or beads). For example, one can utilize an antibody specific for the polynucleotide binding protein in combination with a secondary antibody that is HRP-linked to provide a detectable signal. While the examples described herein present immunoassays for labeling and detection, the present invention can also utilize non-immuno based labeling and detection methods. Other possible labels are optical (emitting) labels, electrostimulatory labels, electromagnetic labels, enzyme conjugated labels such as beta-galactosidase label, and affinity labels such as His-tag and GTS-tag.

In another aspect, the present invention provides methods of screening agents that inhibit or, alternatively, enable or enhance the sequence-specific binding of a polynucleotide binding protein, or fragment thereof, the methods comprising: providing a substrate with a plurality of a polynucleotide fragment bound to its surface; contacting the substrate with an amount of a candidate agent; contacting the substrate with a plurality of a sequence-specific polynucleotide binding protein, or fragment thereof, wherein the sequence-specific polynucleotide binding protein binds at least a portion of the polynucleotide fragment bound to the surface; contacting the substrate with a first antibody specific for the sequence-specific polynucleotide binding protein; contacting the substrate with a detector antibody specific for the first antibody to effect an immunoreaction, the detector antibody comprising a label that provides a detectable signal; detecting the detectable signal; and quantifying the detectable signal relative to a reference signal generated by a control in the absence of the candidate agent to determine the amount of the sequence-specific polynucleotide binding protein, or fragment thereof, bound to the polynucleotide fragment. Reduced detection of the detectable signal relative to a control in the absence of the candidate agent indicates the candidate agent inhibits the sequence-specific binding of the polynucleotide binding protein; increased detection of the detectable signal relative to the control in the absence of the candidate agent indicates the candidate agent enhances or enables the sequence-specific binding of the polynucleotide binding protein.

In some embodiments, reduced detection of the detectable signal relative to a control in the absence of the candidate agent indicates the candidate agent is an agent that binds to the polynucleotide fragment and inhibits the binding of the polynucleotide binding protein thereto. Furthermore, the candidate agent and the sequence-specific polynucleotide binding protein can be contacted together before contacting the substrate such that reduced detection of the detectable signal relative to a control (in the absence of the candidate agent) indicates the candidate agent binds to the polynucleotide binding protein and disrupts the sequence-specific binding of the polynucleotide binding protein to the polynucleotide fragment. In such embodiments, the candidate agent causes a protein conformation change that disrupts the sequence-specific binding of the polynucleotide binding protein to the polynucleotide fragment.

In yet another aspect, the present invention provides methods of screening for agents that enable the sequence-specific binding of a protein, or fragment thereof, to a polynucleotide fragment, the methods comprising: providing a substrate with a plurality of a polynucleotide fragment bound to its surface; contacting the substrate with an amount of a candidate agent; contacting the substrate with a protein, or fragment thereof, that lacks a binding affinity for the polynucleotide fragment; contacting the substrate with a first antibody specific for the protein; contacting the substrate with a detector antibody specific for the first antibody to effect an immunoreaction, the detector antibody comprising a label that provides a detectable signal; detecting the detectable signal; quantifying the detectable signal quantifying the detectable signal relative to a reference signal generated by a control in the absence of the candidate agent to determine the amount of the protein bound to the polynucleotide fragment. Increased detection of the detectable signal relative to a control in the absence of the candidate agent indicates the candidate agent enables the sequence-specific binding of the protein. Furthermore, the candidate agent and the protein, or fragment thereof, can be contacted together before contacting the substrate such that increased detection of the detectable signal relative to a control (in the absence of the candidate agent) indicates the candidate agent binds to the protein, or fragment thereof, and enables the sequence-specific binding of the protein to the polynucleotide fragment. In such embodiments, the candidate agent may cause a protein conformation change that enables the sequence-specific binding of the protein, or fragment thereof, to the polynucleotide fragment.

In some embodiments of aspects described herein, the detectable signal (and/or reference signal) is generated by enzyme-linked immunosorbent assay (ELISA). In additional embodiments, the methods include performing additional assays to confirm that a candidate agent that is isolated in the screening methods described herein performs the targeted protein-nucleic acid interaction as indicated by the screen. Exemplary assays include Western blot, competition dialysis assays, in-cell Western assays, and Oil Red O staining.

In another aspect, the present invention provides enzyme linked immunosorbent assay (ELISA) kits for screening for agents that inhibit, or alternatively enable, the sequence-specific or nonspecific binding of a polynucleotide binding protein, or fragment thereof, the kit comprising: a substrate for binding a polynucleotide fragment of interest; a known amount of a polynucleotide binding protein, or fragment thereof; a known amount of a first antibody specific to the polynucleotide binding protein; and a known amount of a labelled-secondary antibody specific to the first antibody.

The antibodies utilized in the methods and kits of the present invention include full-length antibodies and/or antibody fragments.

In some embodiments, the components of the kits of the present invention are provided in individual containers with known amounts or weights therein. Instructions for use may also be included.

In some embodiments, the polynucleotide fragment of interest is bound to the substrate. In some embodiments, the substrate is a multi-well plate, such as for example, but not limited to, 96, 384, and 1536 well plates. In some embodiments, the kit further comprises a blocking agent.

In some embodiments, the polynucleotide fragment of interest is biotin-labeled and streptavidin is bound to the substrate such that the polynucleotide sequence of interest binds to the substrate via a biotin-streptavidin linkage. Alternatively, the polynucleotide fragment of interest is covalently linked to the substrate.

In some embodiments, the screening kits are adapted to identify inhibitors or activators that target protein-DNA complexes or protein-RNA complexes for medical or other purposes. The inhibitors and activators can be chemical compounds, proteins, DNA molecules, or RNA molecules.

For example, and in no way limiting, screening kits to identify inhibitors or activators targeting HMGA2-DNA complexes are provided. Such screening kits can include the following components: pure HMGA2, a multi-well plate coated with a DNA molecule containing a HMGA2 binding site, such as FL814, 3% BSA in 2×SSCT, the first antibody against HMGA2, the second HRP-linked antibody recognizing the first antibody, a HRP substrate (such as colorimetric ELISA substrates, Chemiluminescent ELISA substrates, or fluorescent ELISA substrates) and sufficient amount of 2×SSCT. These kits can also be used for different purposes. For example, the screening kits described herein can be used as the primary screening assay to screen compound libraries for anticancer drugs or antibiotics targeting a protein-DNA interaction in a high throughput format (drug discovery tools). The screening kits described herein can also be used to determine whether a protein is able to activate or stimulate the binding of a DNA-binding protein to different DNA binding sequences for research purposes. Additionally, the screening kits can be used to identify inhibiting or activating protein factors for a DNA-binding protein in cell extracts (research tools). Likewise, similar embodiments can be utilized for RNA-binding proteins.

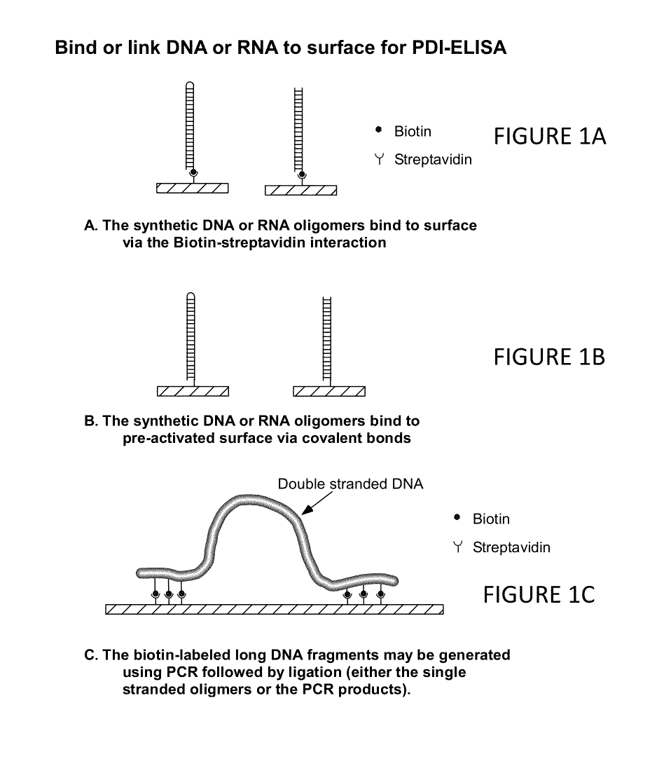

In embodiments of the methods and kits of the present invention, the polynucleotide fragment includes a DNA oligomer, an RNA oligomer, double stranded DNA, double stranded RNA, single stranded DNA, or single stranded RNA. Furthermore, FIGS. 1A-1C illustrate methods that can be utilized in embodiments of the present invention for binding or linking polynucleotide (e.g., DNA) oligomers or fragments to substrate surfaces, e.g., wells of plates or other solid surfaces: the synthetic biotin-labeled oligomers (hairpins or double stranded oligomers or single stranded oligomers) can bind to streptavidin-coated substrates (FIG. 1 (A)); the synthetic oligomers can be covalently linked to pre-activated substrate surfaces (FIG. 1 (B)); and the long biotin-labeled DNA fragments can be generated using PCR followed by DNA ligation and binding to streptavidin molecules on the substrate surface (FIG. 1 (C)).

As used herein, a “polynucleotide molecule” is a biopolymer composed of 2 or more nucleotide monomers covalently linked in a chain, such as DNA (deoxyribonucleic acid) and RNA (ribonucleic acid). A polynucleotide molecule can be single stranded or double stranded.

As used herein, “DNA-binding proteins” are proteins that bind to the double or single stranded DNA in vitro and in vivo, and participate in forming protein-DNA complexes. The following is a list of non-limiting examples of DNA-binding proteins and corresponding DNA-binding sequences that may be utilized in the methods and kits of the present invention: the mammalian high mobility group AT hook-2 (HMGA2); DNA-binding sequences: 5′-ATATTCGCGAWWATT-3′ and 5′-ATATTGCGCAWWATT-3′, where W represents A or T; other AT-rich DNA sequences;

the transcription factor P53 tumor suppressor protein; DNA-binding sequences: 5′-RRRC(A/T)(T/A)GYYY-3′ where R is a purine, Y is a pyrimidine, and N is an unspecified base; the transcription factor nuclear factor KB (NFκB); DNA-binding sequence: 5′-GGGRNYYYCC-3′ where R is a purine, Y is a pyrimidine, and N is an unspecified base;
the transcription factor Sp1; DNA-binding sequence: 5′-(G/T)GGGCGG(G/A)(G/A)(C/T)-3′ (GC box element);
the transcription factor, the activator protein 1 (AP-1); DNA-binding sequence: 5′-TGA(G/C)TCA-3;
the transcription factor CCAAT-enhancer-binding proteins (C/EBPs); DNA-binding sequence: 5′-ATTGCGCAAT-3′;
the transcription factor, Heat shock factor (HSF); DNA-binding sequence: 5′-NGAAN-3′ where N is an unspecified base;
the transcription factor ATF/CREB; DNA-binding sequence: 5′-TGACGTCA-3′;
the transcription factor c-Myc; DNA-binding sequence: 5′-CACGTG-3′;
the transcription factor Oct-1; DNA-binding sequence: 5′-ATGCAAAT-3;
the transcription factor Nuclear factor I (NF-I); DNA-binding sequence: 5′-TTGGCNNNNNGCCAA-3′ where N is an unspecified base;
the lac repressor (LacI or LacR); DNA-binding sequences: the lac Os, O1, O2, and O3 operators;
the gal repressor (GalR); DNA-binding sequence: the gal OI and OE operators;
the coliphage lambda cI protein; DNA-binding sequence: the bacterial phage lambda operators; and
DNA-binding proteins involved in DNA replication, recombination, transcription, and repairs.

As used herein, “RNA-binding proteins” are proteins that bind to the double or single stranded RNA in vitro and in cells and participate in forming ribonucleoprotein complexes. The following is a list of non-limiting examples of RNA-binding proteins that may be utilized in the methods and kits of the present invention: proteins in ribosomes; proteins involved in RNA splicing, RNA editing, RNA export, mRNA localization, translation, and microRNA recognition. Embodiments of the present invention can also be used as a guiding or screening tool for the synthesis of DNA-binding compounds or the production of DNA-binding proteins (motifs or domains). These DNA-binding compounds and DNA-binding proteins can be sequence-specific or nonspecific. For example, a screening kit can include a pure non-specific DNA-binding protein, such as E. coli HU protein or mammalian histone H1, a multi-well plate coated with a DNA molecule containing a targeted DNA-binding site, the first antibody against HU or histone H1, the second HRP-linked antibody recognizing the first antibody, and a HRP substrate, such as colorimetric ELISA substrates, Chemiluminescent ELISA substrates, or fluorescent ELISA substrates. Similar screening kits can be adapted for RNA-binding compounds or proteins. It is expected that the signal to noise ratio of the kits and methods described herein will be much higher than that of the commonly used ethidium displacement assays or acridine orange displacement assays. The screening assays are much more robust as well.

### EXAMPLES

The methods and related kits herein described are further illustrated in the following examples, which are provided by way of illustration and are not intended to be limiting. It will be appreciated that variations in proportions and alternatives in elements of the components shown will be apparent to those skilled in the art and are within the scope of embodiments of the present invention. Theoretical aspects are presented with the understanding that Applicants do not seek to be bound by the theory presented. All parts or amounts, unless otherwise specified, are by weight.

The following Materials and Methods were used for all the methods and related kits exemplified herein.

### Materials

Biotin-labeled hairpin DNA oligomer FL814 containing a specific binding site of HMGA2 was purchased from Eurofins MWG Operon, Inc. Streptavidin covalently coated 96-well plates (NUNC Immobilizer Streptavidin-F96 clear) were from Thermo Fisher Scientific, Inc. Antibody against HMGA2 (HMGA2 (D1A7) Rabbit mAb) and Anti-rabbit IgG, HRP-linked Antibody #7074 were purchased from Cell Signaling, Inc. Ultra TMB-ELISA was bought from Thermo Fisher Scientific, Inc. The mammalian high mobility group protein AT hook 2 (HMGA2) was purified as described previously (Cui, T., et al. (2007)). Netropsin, insulin and Oil red O were purchased from Sigma and used without further purification. The following extinction coefficients were used to determine the concentration of different compounds: netropsin, 21 500 M-¹ cm-¹ at 296 nm, meso-tetra(N-methyl-4-pyridyl) porphine tetratosylate (TMPyP4), 226 000 M-¹ cm-¹ at 424 nm and HMGA2, 5810 M-¹ cm-¹ at 280 nm. A compound library consisting of 29 DNA-binding compounds was a generous gift of Prof. Jonathan B. Chaires (University of Louisville, Ky., USA). Dulbecco's modified Essential Medium (DMEM) and fetal bovine serum (FBS) were purchased from Invitrogen, Inc.

### Protein-DNA Interaction ELISA Assays to Screen Compounds Targeting HMGA2-DNA Interactions

In this method, the first step is to bind a biotin-labeled oligomer to a streptavidin-coated 96-well plate. A synthetic DNA hairpin oligomer FL814 carrying a specific binding site of HMGA2, SELEX1, was used. The DNA oligomer was dissolved into an annealing buffer (10 mM Tris-HCl pH 8.0, 50 mM NaCl) at 100 μM and heated in a water bath to 95° C. for 10 min. The denatured DNA oligomer FL814 was cooled down slowly for the formation of the doublestranded DNA. The streptavidin-coated plate was washed three times with 300 μl of 2×SSCT (saline-sodium citrate buffer with Tween 20:30 mM trisodium citrate pH 7.0, 200 mM NaCl and 0.05% Tween 20). After the wash, 100 μl of 0.1 μM FL814 was added to each of the wells. The plate was then incubated at room temperature on a shaking platform for 1 h. After removing the DNA solution, the plate was washed three times with 300 μl of 2×SSCT. In the next step, 300 μl of 3% bovine serum albumin in 2×SSCT was added to each of the wells to block the surface overnight at 4° C. The plate was then washed three times with 300 μl of 2×SSCT. The next step was the binding of HMGA2 to the DNA on the well surface of the 96-well plates. A titration of various concentrations of HMGA2 was carried out for the determination of the optimal signal-to-noise ratio for the assay. In this step, the DNA-binding compounds can be added to the wells to inhibit or enhance HMGA2 binding to the DNA oligomer FL814. After the protein binding step, the plate was washed three times with 300_l of 2×SSCT. One hundred microliter of the first antibody against HMGA2 (Rabbit mAb) in 2×SSCT was added to the wells and the plate was then incubated at room temperature for 1 h on a shaking platform. The plate was washed three times with 300 μl of 2×SSCT. One hundred microliter of the second antibody against HMGA2 (HRP-linked anti-Rabbit IgG) in 2×SSCT was added to the wells and the plate was then incubated at room temperature for 1 h on a shaking platform. The plate was washed three times with 300 μl of 2×SSCT. After the wash, 100 μl of Ultra TMB ELISA was added to the wells. The plate was then incubated at room temperature for 15 min on a shaking platform. The reaction was then stopped with 100 μl of 2MH2SO4. The results were quantified with photometric detection using a microplate reader. The protein-DNA interaction enzyme-linked immunosorbent assays (PDI-ELISA) were also used to determine the apparent DNA dissociation constant (Kd) by nonlinear-least-squares fitting the following equation using the program Origin:

$$R = \frac{( a + x + {K}_{d} ) - \sqrt{{( a + x + {K}_{d} )}^{2} - 4 ax}}{2 a} ,$$

where R, a and x represent the DNA-binding ratio, the total DNA concentration and the total protein concentration, respectively. The apparent inhibitory IC50 values were obtained using the following equation (Miao, Y, et al. (2008)):

%HMGA2 binding to DNA=100/[1+C*(1+K_{c2}*C)/[IC₅₀(1+K_{c2}*IC₅₀)]],

where K_{c2} is a macroscopic binding constant for inhibitor binding to DNA, IC₅₀ is the concentration of inhibitor that causes 50% inhibition of HMGA2 binding to DNA and C is the concentration of an inhibitor.

Z-factor of the PDI-ELISA was determined using a full 96-well plate in which 48 wells are for positive controls in the presence of 1 μM of HMGA2 and the rest 48 wells for negative controls in the absence of HMGA2. Z-factor was calculated by the following formula:

$$Z = 1 - \frac{3 ( {σ}_{p} + {σ}_{n} )}{\vert {μ}_{p} - {μ}_{n} \vert} ,$$

where σp, σn, μp and to represent the sample means and standard deviations for positive (p) and negative (n) controls, respectively.

### Competition Dialysis Assays

The competition dialysis assays were carried out according to previously published procedures (Ren, J. and Chaires, J. B. (1999)). Briefly, a volume of 0.3 ml of FL814 (3 μM) or the HMGA2-FL814 complex (3 μM) was pipeted into a separate 0.3 ml disposable dialyzer. The dialysis units were then placed into a beaker with 200 ml of 2×SSCT containing 0.73 μM of TMPyP4. The dialysis was allowed to equilibrate with continuous stirring of 72 h at room temperature (24° C.). After the dialysis, the free, bound and total concentrations of TMPyP4 were determined spectrophotometrically.

### Pre-Adipocytes and Differentiation

Mouse pre-adipocytes (3T3-L1 cells) were a gracious gift from Dr Jun Liu at Mayo Clinic. 3T3-L1 cells were maintained in DMEM supplemented with 10% FBS, 100 U/ml penicillin, 100 μg/ml streptomycin and 5 μg/ml plasmocin. The cells were grown under normal culture conditions of 5% CO₂ and humidity. Cells were maintained for a minimum of three passages in linear growth prior to differentiation. To differentiate the 3T3-L1 pre-adipocytes into adipocytes, we used the protocol previously described by Zebisch et al. (Zebisch, K., et al. (2012)). Briefly, 3T3-L1 pre-adipocytes were sub-cultured in DMEM supplemented with 5% newborn calf serum, 100 U/ml penicillin, 100 μg/ml streptomycin and 5 μg/ml plasmocin (Basal Media 1—BM1). The cells were then plated at 6×10⁵ cells per dish in 35-mm dishes, 3×10⁵ cells per well in a 12-well plate, 1.5×10⁵ cells in a 24-well plate or 3.0×10⁴ cells per well in a 96-well plate. The cells were grown to confluency with BM1 replacement every other day. Following 48 h of confluency, 3T3-L1 cells were treated with Differentiation medium 1 (DM1), which is BM1 supplemented with 0.5 μM iso-butyl-methylxanthine, 0.25 μM dexamethasone, 1 μg/ml insulin and 2 μg/ml rosiglitazone. After 48 h, the medium was replaced with differentiation medium 2 (DM2); this is BM1 supplemented with 1 μg/ml insulin. On the seventh day, the cells were placed in DMEM with FBS. The cells were cultured to the date indicated in the experiments below with media changes every other day. The presence of adipogenesis was confirmed by microscopic detection of adipose bodies in the cells. For our experiments, only cultures with >80% differentiation were used.

### Western Blot Analysis

To isolate proteins from cells for western blot analysis, cells were plated as indicated above, and following differentiation and drug treatment, cells were lysed and proteins were harvested as previously described (Chambers, J. W., et al. (2013)). Briefly, cells were washed twice in phosphate buffered saline (PBS) and lysed in radioimmunoprecipitation assay buffer (50 mM Tris-HCl, pH 8.0, 150 mM NaCl, 1% Nonidet P-40, 0.5% deoxycholate, 0.1% SDS) supplemented with protease inhibitors (1 mM PMSF and Halt Protease Inhibitor Cocktail (Thermo)) and Halt phosphatase inhibitors (Thermo). Cells were then incubated at 4° C. for 5 min with gentle rocking Cells were scraped from the well and transferred to a sterile micro-centrifuge tube. Following a 10-min incubation on ice, the cells disruption was finished using sonication. The lysate was centrifuged at 14 000×g for 15 min to remove debris. Protein concentrations of the supernatant were determined using the Pierce BCA Assay kit protocol. Proteins were resolved by sodium dodecyl sulphatepolyacrylamide gel electrophoresis and transferred onto polyvinylidene difluoride membranes. Membranes were incubated with Li-Cor Biosciences Odyssey Blocking for at least 1 h at room temperature or overnight at 4° C. The membranes were incubated with primary antibodies specific for FABP4, HMGA2 and Actin (Cell Signaling Technology 3544, 5269 and 3700, respectively) at dilutions of 1:1000 in blocking buffer. Membranes were washed three times for 5 min in 1×TBST (20 mM Tris-HCl, pH 6.7, 137 mM NaCl, 0.1% Tween 20). Membranes were incubated with secondary antibodies in blocking buffer at 1:20 000 for fluorescently conjugated antibodies purchased from Li-Cor Biosciences. Membranes were again washed three times for 5 min in 1×TBST. Western blots were developed using fluorescence detection using the LI-COR Odyssey CLx near infrared scanner.

### Oil Red O Staining

Following differentiation and drug treatment, Oil Red O staining was used to qualitatively and quantitatively assess the level of neutral lipids in the differentiated cells. For qualitative assessment by phase microscopy, cells were washed twice in PBS and then fixed with 10% formalin for 30 min at room temperature. Oil Red O was reconstituted at 300 mg per 100 ml of isopropanol. The Oil Red O solution was prepared for staining by adding 20 ml of deionized water to 30 ml of the Oil Red O stock solution and filtered. The cells were then incubated for 5 min in 60% isopropanol. The Oil Red O working solution was then added to cells for 5 min. The monolyers were then rinsed clean with deionized water. The cells were then visualized using phase microscopy on the EVOS XL Core microscope. For quantitative assessment of adipogenesis in 96-well formats, cells were lysed in dimethylsulfoxide (DMSO) following the water wash of Oil Red O stained cells. The DMSO released the intracellular Oil Red O into the solution. The absorbance (492 nm) was measured using the BioTek Synergy H1 plate reader.

### In-Cell Western Analysis

Specific protein levels were analyzed using in-cell western technology (Egorina, E. M., et al (2006)). During differentiation and drug administration, 3T3-L1 cells were fixed in 4% paraformaldehyde in PBS for 25 min at room temperature. Pre-adipocytes were quenched by washing the cells in 100 mM glycine for 5 min at room temperature. The cells were permeabilized by incubating the cells in 0.2% Triton X-100 in PBS for 30 min at room temperature. The cells were then blocked using LICOR Odyssey Blocking Buffer (LI-COR Biosciences, 927-40100) for 1 h at room temperature. Cells were then incubated with a primary antibody for HMGA2 (Cell Signaling Technologies #5269) at a 1:500 dilution in LI-COR Odyssey Blocking Buffer supplemented with 0.1% Tween-20 for 2.5 h at room temperature. The cells were washed in 0.1% Tween in PBS. Secondary antibodies (IRDye 800CW anti-rabbit, LI-COR Biosciences, 926-32211) were incubated at a dilution of 1:1000 in LI-COR Blocking Buffer supplemented with 0.1% Tween-20 for 1 h. Concurrent to the addition of secondary antibody TO-PRO-3 (Invitrogen) was added at a 1:1000 dilution as a normalization control for cell number and incubated for 1 h. The cells were washed again in 0.1% Tween-20 in PBS. Plates were then scanned on the LICOR Biosciences Odyssey CLx Infrared Imaging System and quantified using the LI-COR in-cell western analysis in Image Studio.

### Statistical Analysis

A minimum of eight technical replicates was considered for all cell-based studies performed in 96-well formats, while technical replicates were performed for western blot analysis and microscopy; additionally, a minimum of four biological replicates were evaluated for our studies. To determine statistical significance, Student's paired t test was employed for significance between treatments. Statistical significance is indicated by an asterisk in figures in which the P value is <0.05. Data are displayed as means with error bars representing plus and minus one standard deviation.

### Example 1

### High Throughput Screening for Compounds that Block DNA-Binding Proteins

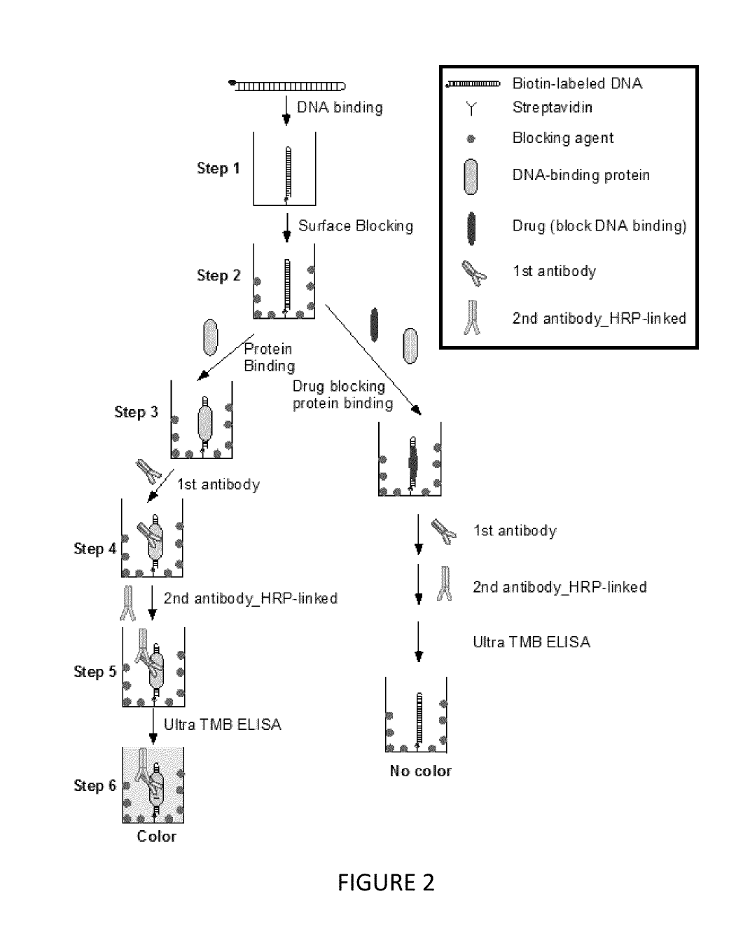

FIG. 2 illustrates a high throughput drug screening using the methods described herein in which PDI-ELISA is utilized (Hibma, M. H., et al. (1994); Rosenau, C., et al. (2004); Brand, L. H., et al. (2013)). The first step is to bind a biotin-labeled oligomer to the streptavidin-coated multiple-well plates (e.g., 96, 384, or 1536 well plates). Either a synthetic hairpin or double-stranded DNA oligomer containing a binding sequence for a sequence-specific DNA-binding protein, such as transcription factors, may be used. The DNA oligomer is dissolved into an annealing buffer (10 mM Tris-HCl, 50 mM NaCl) at 50 or 100 μM and heated in a water bath or thermocycler to 95° C. for 10 minutes. The denatured DNA oligomers are cooled down slowly for the formation of the double-stranded DNA. The streptavidin coated 96, 384, or 1536 well plates can be purchased from various companies, such as Thermo Fisher Scientific, Inc. Although 96, 384, or 1536 well plates non-covalently coated with streptavidin could be used for the assays, it is preferable to use 96, 384, or 1536 well plates coated with streptavidin covalently. The streptavidin-coated 96, 384, or 1536 well plates are prewashed with 3×300, 3×100, or 3×10 μL/well of either TBST (Tris-Buffered Saline with Tween 20; 50 mM Tris-HCl, pH 8, 150 mM NaCl, and 0.05% Tween 20) or 2×SSCT (saline-sodium citrate buffer with tween 20; 30 mM trisodium citrate pH 7.0, 300 mM NaCl, and 0.05% Tween 20), respectively. After the wash, 100, 50, or 3 μL of biotin-labeled DNA oligomer (0.1 or 0.2 μM in either TBST or 2×SSCT) is added to the wells of the streptavidin-coated 96, 384, or 1536 well plates, respectively, and incubated at room temperature for 1 hour.

After the 1-hour incubation, the DNA coated 96, 384, or 1536 well plates are washed with 3×300, 3×100, or 3×10 μL/well of TBST or 2×SSCT. The second step is to block the well surface by 3% BSA or 5% non-fat dry milk (300, 100, or 10 μL in TBST or 2×SSCT) for either 1 hour or overnight at 4° C. After the blocking, the plates are washed three times with TBST or 2×SSCT. The third step is the binding of the DNA-binding proteins to the DNA on the well surface of the 96, 384, or 1536 well plates. A titration of various concentrations of the DNA-binding protein should be carried out for the determination of the optimal signal to noise ratio for the assay. In this step, the DNA-binding compounds can be added to the well to either inhibit or activate or enhance the protein binding to the DNA or RNA molecule. After the binding, the plates are washed three times with TBST or 2×SSCT.

In the next few steps, the first antibody recognizing the DNA-binding protein is added to the well; and after washing with 3×300, 3×100, or 3×10 μL/well of TBST or 2×SSCT, the second HRP-linked antibody recognizing the first antibody is added to the well. After washing three times with TBST or 2×SSCT, 100, 50, or 3 μL of a HRP substrate, such as colorimetric ELISA substrates, Chemiluminescent ELISA substrates, or fluorescent ELISA substrates, is added to the wells. Absorbance, the chemiluminescence signal (relative light units, RLU), or fluorescence signal can be read by a microplate reader to determine the extent by which compounds inhibit or enhance the DNA-binding capacities of the DNA-binding protein. In the presence of sufficient amount of a DNA-binding compound, only the background signal for the inhibitors should be detectable; the activators should also be detectable. The apparent inhibitory or stimulatory IC₅₀ of the DNA-binding compound can be determined as well.

The same methods can be used to screen compounds against RNA-binding proteins.

### Example 2

### High Throughput Screening for Compounds that Bind to DNA-Binding Proteins

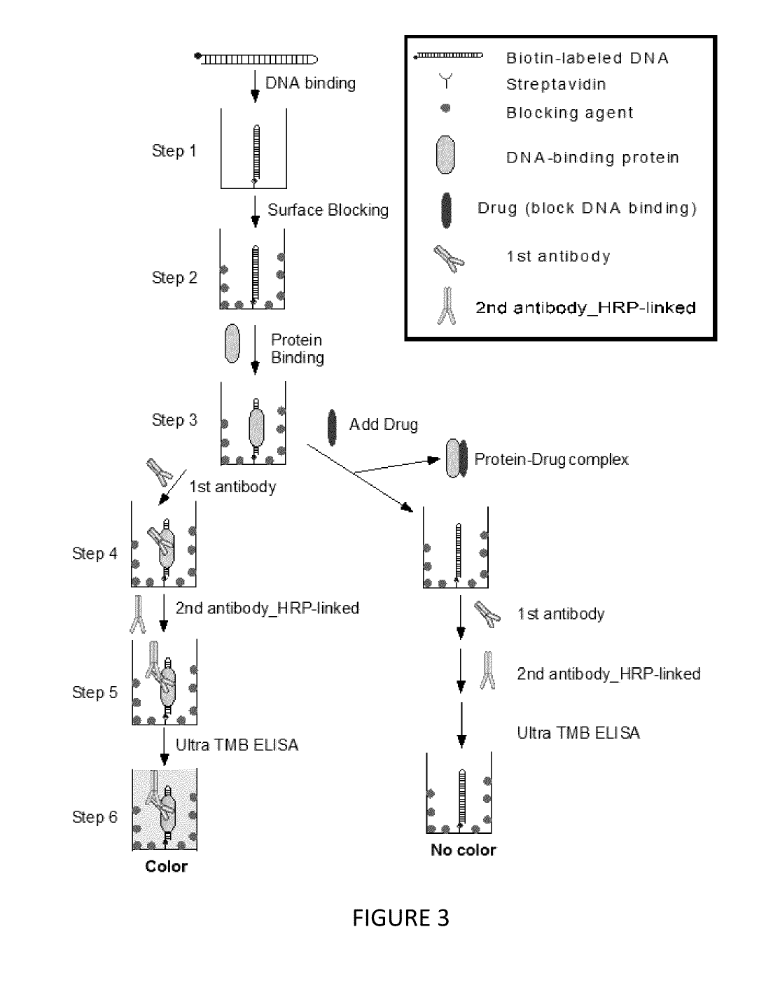

FIG. 3 shows another embodiment of a method for high throughput compound screening using PDI-ELISA. In this embodiment, compounds are screened that specifically bind to the DNA-binding protein and block the protein binding to the DNA molecule. The same methods can be used to screen compounds against RNA-binding proteins.

### Example 3

### High Throughput Screening for Compounds that Bind Proteins and Cause Sequence-Specific DNA-Binding

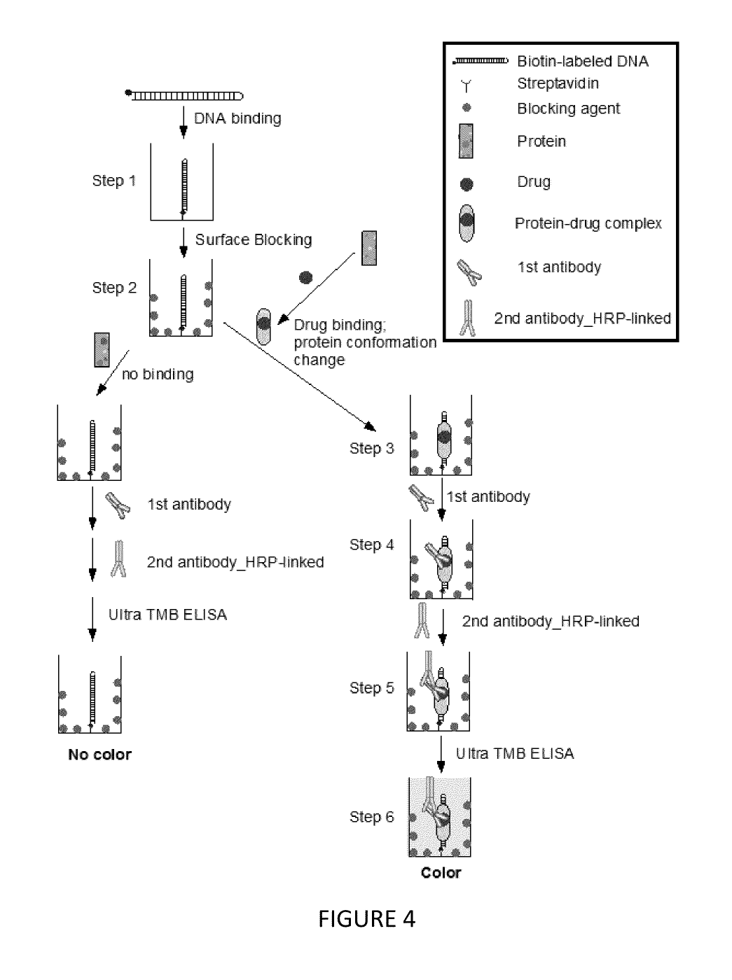

FIG. 4 shows another embodiment of a method for high throughput compound screening using PDI-ELISA. In this embodiment, compounds that bind to a protein and cause protein conformational change are screened. For example, the compound-protein complex becomes a sequence-specific DNA-binding protein and binds to the DNA oligomer. The same methods can be used to screen compounds against RNA-binding proteins.

### Example 4

### High Throughput Screening for Compounds that Block DNA-Binding Proteins

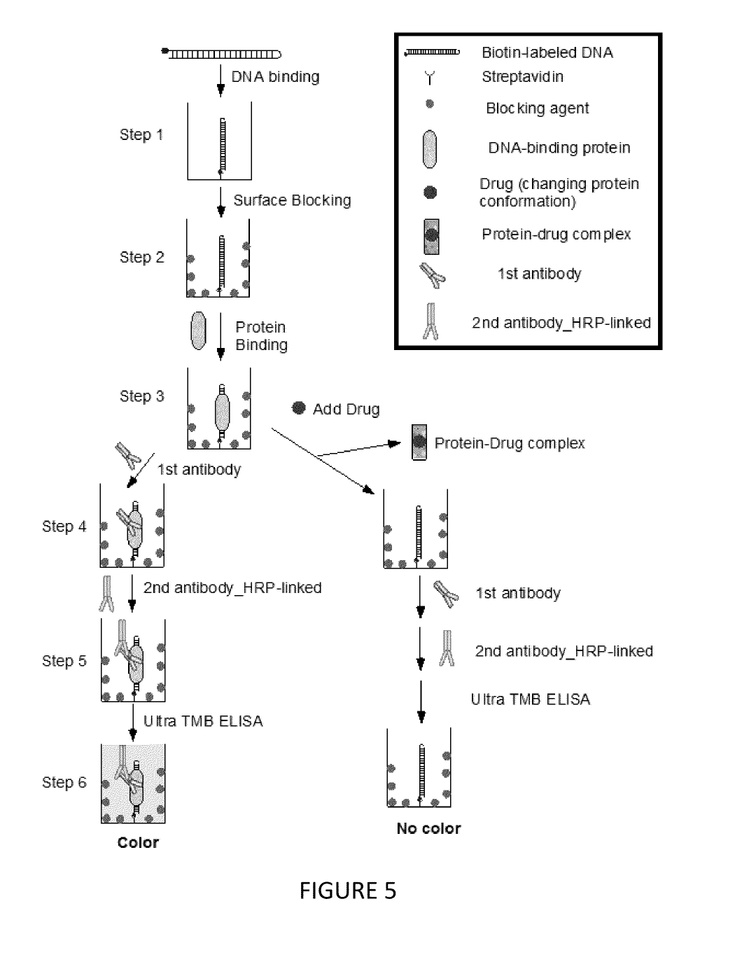

FIG. 5 shows another embodiment of a method for high throughput compound screening using PDI-ELISA. In this embodiment, a small molecule binds to the DNA-binding protein and causes the conformational change of the protein. In this case, the protein no long binds to its binding site on the DNA oligomer. The same methods can be used to screen compounds against RNA-binding proteins.

### Example 5

### High Throughput Screening for Compounds that Bind DNA Molecules and Stimulate/Enhance DNA Binding Protein Interactions

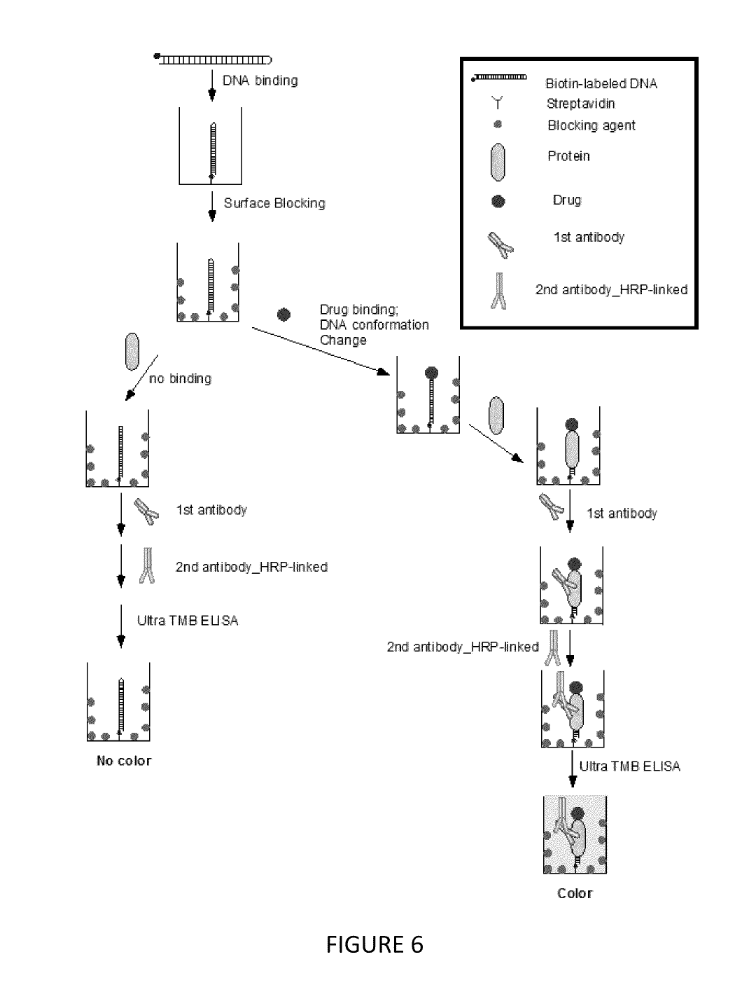

FIG. 6 shows another embodiment of a method for high throughput compound screening using PDI-ELISA. In this embodiment, a small molecule binds to the DNA molecule and causes the conformational change of the DNA molecule. In this scenario, the protein is able to bind to the target DNA molecule or the DNA binding capacity of the protein molecule gets significantly enhanced or stimulated. The same methods can be used to screen compounds against RNA-binding proteins.

### Example 6

### Drug Screening Utilizing HMGA2

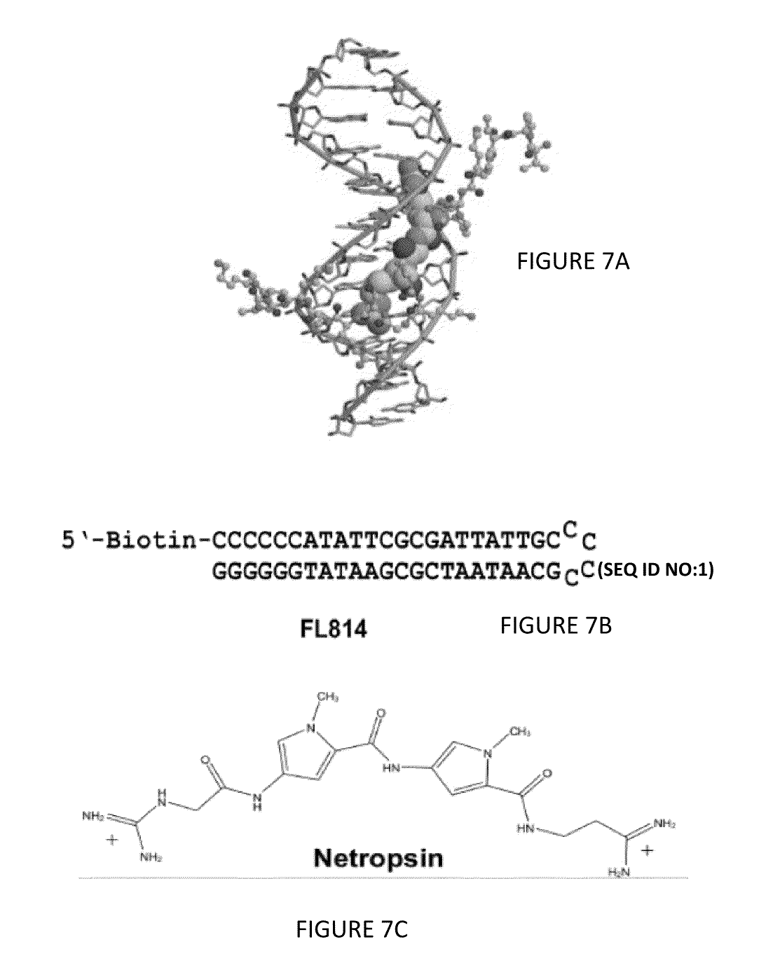

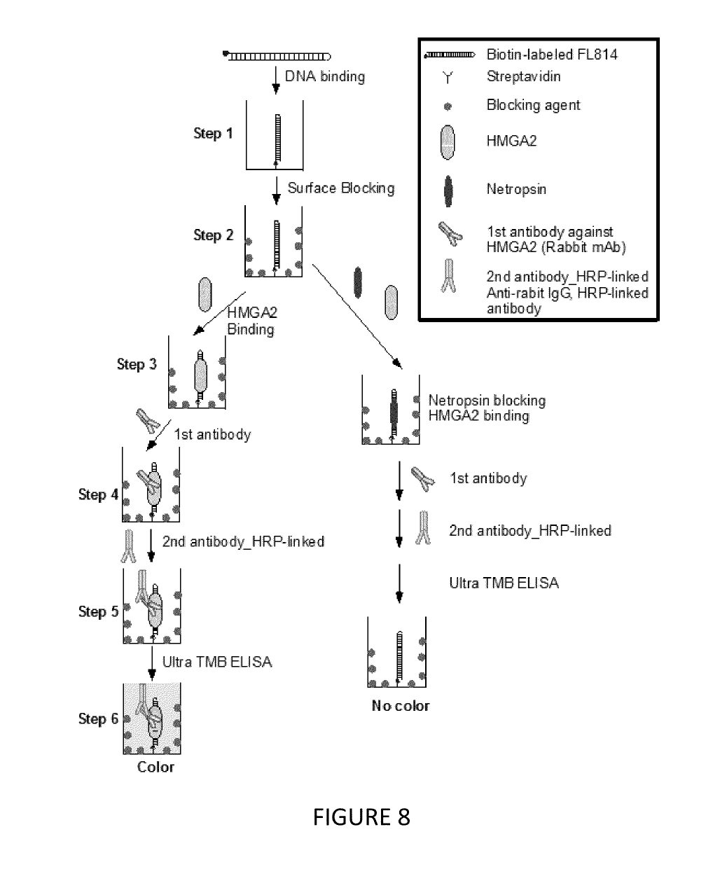

The methods described in EXAMPLE 1 and EXAMPLE 5 were utilized with the mammalian high mobility group protein AT-hook 2 (HMGA2) as the sequence-specific DNA-binding protein. HMGA2 is a multi-function nuclear transcription factor directly linked to oncogenesis (Young, A. R. & Narita, M. (2007); Morishita, A., et al. (2013)) and obesity (Zhou, X., et al. (1995); Anand, A. & Chada, K. (2000)). It is also involved in human height (Weedon, M. N., et al. (2007); Horikoshi, M., et al. (2013)), stem cell youth (Copley, M. R., et al. (2013)), and human intelligence (Stein, J. L., et al. (2012)). These functionalities make HMGA2 a good candidate for utilization in drug screening (Miao, Y, et al (2008); Fusco, A. and Fedele, M (2007)). HMGA2 is a small DNA-binding protein carrying three “AT hook” DNA binding motifs that specifically recognize the minor groove of AT-rich DNA sequences (Cui, T. & Leng, F. (2007)) (FIG. 7 (A)). A biotin-labeled oligomer (a hairpin) FL814 containing a specific binding site of HMGA2, SELEX 1 (FIG. 7 (B); (Miao, Y., et al. (2008)) from Eurofins MWG Operon, Inc. was used in addition to colorimetric detection assays. Streptavidin covalently coated 96 well plates (NUNC Immobilizer Streptavidin-F96 clear) from Thermo Fisher Scientific, Inc. (Catalog number, 436014) were also used. FIG. 8 shows the procedure of the PDI-ELISA for HMGA2 binding to FL814 and the inhibition of HMGA2 binding by netropsin, a minor groove binding drug recognizing AT-rich DNA sequences (FIG. 7 (C)). First, 0.2 μM of FL814 in 2×SSCT was prepared and the 96 well plates prewashed with 3×300 μL/well of 2×SSCT. Then, a solution of 0.2 μM FL814 was added in 2×SSCT to the wells of the Immobilizer Streptavidin plate (100 μL/well) and the plate incubated with gentle agitation for 1 hour at room temperature. The wells were aspirated and washed with 3×300 μL/well of 2×SSCT. The surface of the wells was blocked using 3% BSA in 2×SSCT overnight at 4° C. and washed with 3×300 μL/well of 2×SSCT. Afterwards, a titration experiment was performed by adding various concentrations of HMGA2 to the wells coated with FL814. 100 μL/well of the first antibody against HMGA2 in 3% BSA in 2×SSCT (HMGA2 (D1A7) Rabbit mAb, Cell Signaling; 1:1000 dilution) was added and the plate incubated with gentle agitation for 1 hour at room temperature. The wells were aspirated and washed with 3×300 μL/well of 2×SSCT. And then 100 μL/well of the HRP-linked second antibody against the first antibody, i.e., Anti-rabbit IgG, HRP-linked Antibody #7074 (Cell Signaling, Inc.) in 3% BSA in 2×SSCT was added and the plate incubated with gentle agitation for 1 hour at room temperature. The wells were then aspirated and washed with 3×300 μL/well of 2×SSCT. In the final step, Ultra TMB-ELISA, a chromogenic substrate for horseradish peroxidase (Thermo Fisher Scientific, Inc.) was added at 100 μL/well and the plate incubated with gentle agitation for 15 minutes at room temperature. 100 μL/well of sulfuric acid was added to stop the reaction and the absorbance of each well was measured at 450 nm using a microplate reader.

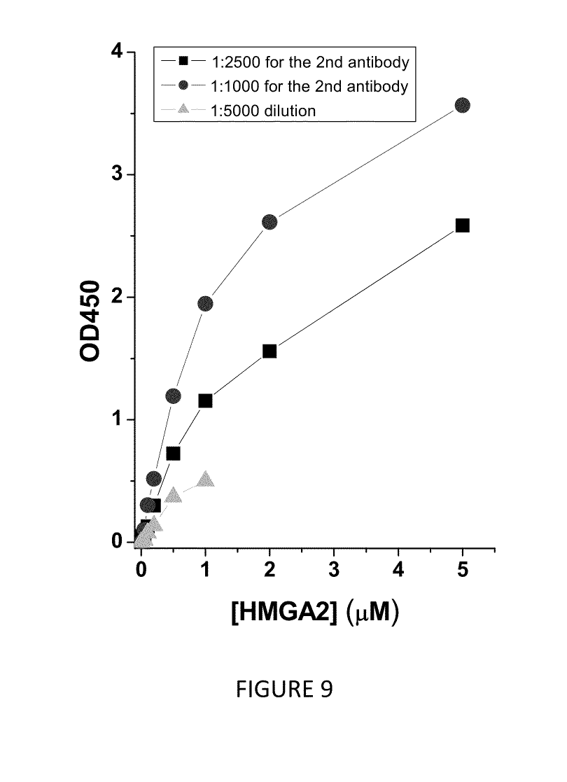

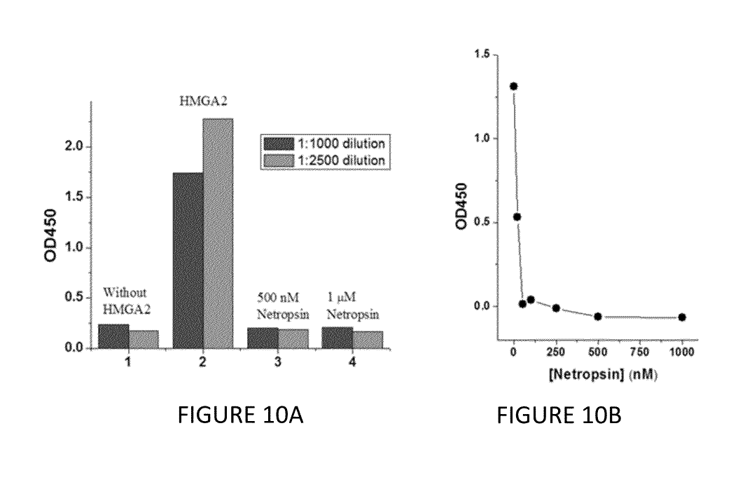

FIG. 9 shows the results of HMGA2 titration experiments where three different dilutions of the second antibody, Anti-rabbit IgG, HRP-linked Antibody (1:5000, 1:2500, and 1:1000 dilutions) were used. The absorbance at OD450 is correlated with the HMGA2 concentration, which is consistent with previously published results (1). FIGS. 10A-10B show the inhibition of HMGA2 binding to FL814 by netropsin. In these two experiments, 0.5 or 1 μM of HMGA2 binding to FL814 was used. For the experiment shown in FIG. 10A, two different dilutions of Anti-rabbit IgG, HRP-linked Antibody (1:1000 and 1:2500 dilutions) was used. In the absence of netropsin, the OD450 reached ˜1.7 and 2.3 for these two dilutions, respectively. In the presence of either 500 nM or 1 μM of netropsin, the absorbance at 450 nm returned to the background level, indicating the complete inhibition of HMGA2-DNA interactions by netropsin. FIG. 10B shows the netropsin titration experiment in which 0.5 IM of HMGA2 was used. Using these results, the inhibitory IC₅₀ of netropsin was determined to be ˜10 nM, which is consistent with previously published results (Miao, Y., et al. (2008)). These experiments demonstrated that methods utilizing ELISA described by the present invention are ready for high throughput drug screening against protein-DNA/RNA interactions.

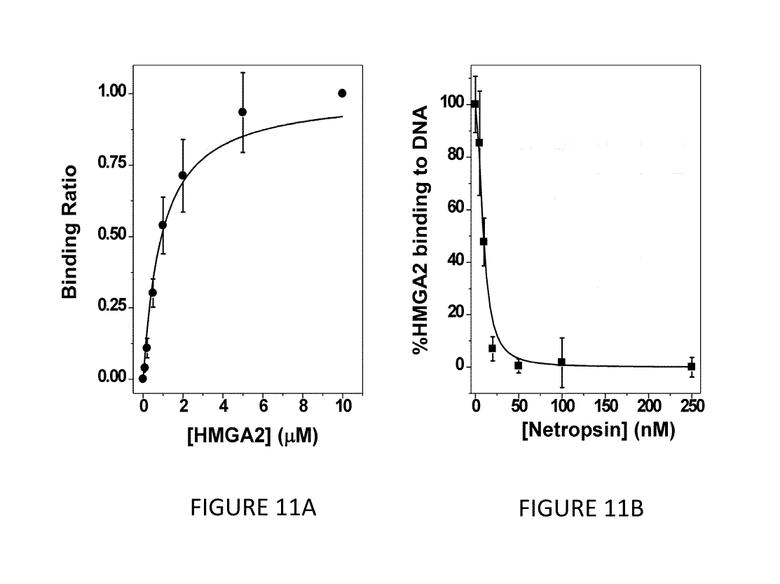

Additionally, the dissociation constant of HMGA2 with DNA FL814 in 2×SSCT was estimated to be ˜1 μM (specifically 0.86±0.12 μM) using PDI-ELISA in which 100 nM of FL814 and the 1:2000 dilution of Anti-rabbit IgG, HRP-linked Antibody were used (FIG. 11A). These results are consistent with the previous published results (Cui, T. & Leng, F. (2007)). Netropsin, a well-characterized AT minor groove binder and a potent inhibitor of HMGA2-DNA interactions (Miao, Y, et al (2008)), was used as a model inhibitor for the proof-of-concept experiment. Results in FIG. 11B demonstrate that netropsin is indeed a strong inhibitor of HMGA2-DNA interactions with an IC₅₀ of 9.30±0.78 nM. Z factors for three consecutive days were determined to be 0.66, 0.68 and 0.77 using the described assay conditions (Materials and Methods) with manual pipetting.

Using the validated screening assay, a collection of 29 different DNA binding compounds were screened against the HMGA2-FL814 interaction. These compounds included 8 DNA intercalators (ethidium bromide, doxorubicin, actinomycin D, Ellipticine, proflavine hydrochloride, proflavine hemisulfate, WP 762, and WP631), 6 DNA minor groove binders (netropsin dihydrochloride, DAPI, berenil, pentamidine isethionate salt, Hoechst 33258, and chromomycin), 6 DNA triplex binders (α-naphthoflavone, β-naphthoflavone, quinacrine mustard dihydrochloride, quinacrine dihydrochloride, coralyne chloride, and berberine chloride), 6 DNA quadruplex binders (N-methyl mesoporphyrin (NMM 580), meso-tetra(N-methyl-2-pyridyl) porphine tetrachloride (TMPyP2), meso-tetra(N-methyl-4-pyridyl) porphine tetratosylate (TMPyP4), thiazole orange, DODC, and N,N′-bis[2-(1-piperidino)ethyl]-3,4,9,10-perylenetetracarboxylic diimide (PIPER)), and 3 miscellaneous DNA binders (1-pyrenemethylamine hydrochloride, methylene blue, and methyl green). 100 nM of drugs were used for the screening assays. For the PDI-ELISA assays where 100 nM of FL814 and 1 μM of HMGA2 were used, in the absence of drugs, OD450 was determined to be 1.44. The following table shows results (OD450 values) in the presence of different DNA-binding compounds.

TABLE 1
The OD450 values of the PDI-ELISA assays
in the presence of different inhibitors.

| Compounds | OD450 | Std |
|---|---|---|
| HMGA2 | 1.438 | 0.069 |
| Ethidium bromide | 1.592 | 0.062 |
| Doxorubican | 1.249 | 0.069 |
| Actinomycin D | 1.03 | 0.031 |
| Ellipticine | 1.39 | 0.049 |
| Proflavine hydrochloride | 1.5 | 0.062 |
| Proflavine hemisulfate | 1.49 | 0.033 |
| WP762 | 0.56 | 0.029 |
| WP631 | 0.21 | 0.013 |
| Netropsin | 0.152 | 0.01 |
| DAPI | 0.161 | 0.011 |
| Berenil | 0.52 | 0.023 |
| Pentamidine | 0.65 | 0.035 |
| Hoechst 33258 | 0.166 | 0.01 |
| Chromomycin | 1.3 | 0.063 |
| a-Naphthoflavone | 1.91 | 0.048 |
| b-Naphthoflavone | 1.56 | 0.026 |
| Quinacrine mustard dihydrochloride | 1.82 | 0.074 |
| Quinacrine dihydrochloride | 1.46 | 0.056 |
| Coralyne chloride | 1.38 | 0.044 |
| Berberine chloride | 0.91 | 0.022 |
| NMM 580 | 1.62 | 0.071 |
| TMPyP2 | 1.7 | 0.059 |
| TMPyP4 | Very high | NA |
| Thiazole orange | 1.83 | 0.047 |
| DODC | 1.3 | 0.039 |
| PIPER | 1.27 | 0.071 |
| 1-Pyrenemethylamine hydrochloride | 1.36 | 0.063 |
| Methylene Blue | 0.93 | 0.035 |
| Methyl Green | 1.06 | 0.041 |

The PDI-ELISA assays were carried out as described under Materials and Methods. The standard deviations (Std) were obtained from three different experiments.

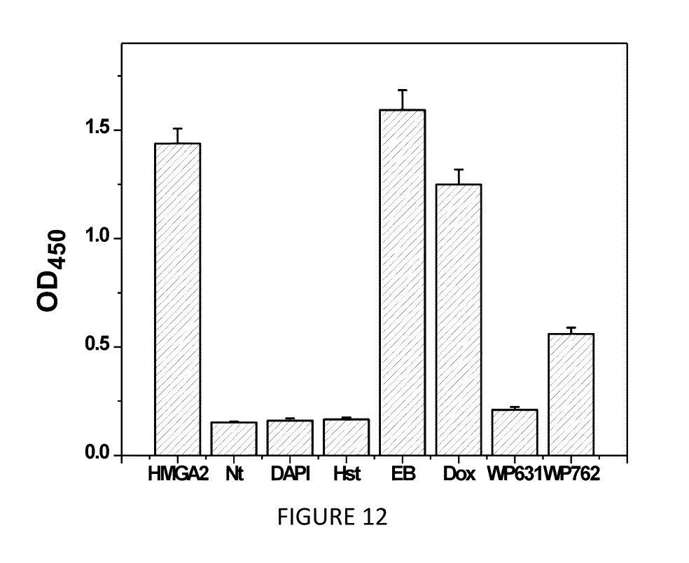

From these values, it was observed that three DNA minor groove binding drugs (netropsin, DAPI, and Hoechst 33258) and two bisintercalators (WP631 and WP62) are able to potently inhibit HMGA2 binding to the target sequence. FIG. 12 shows the inhibition of these results for a few DNA-binding drugs. Table 2 shows the IC₅₀ values of these compounds.

TABLE 2
IC₅₀ values for inhibition of binding of HMGA2 to
DNA by different DNA binding compounds at 25° C.

| Compounds | IC₅₀ (nM) |
|---|---|
| Netropsin | 9.3 ± 0.8 |
| DAPI | 60.6 ± 8.5 |
| Hoechst33258 | 73.0 ± 9.6 |
| WP631 | 41.0 ± 4.5 |
| WP762 | 115.3 ± 19.7 |

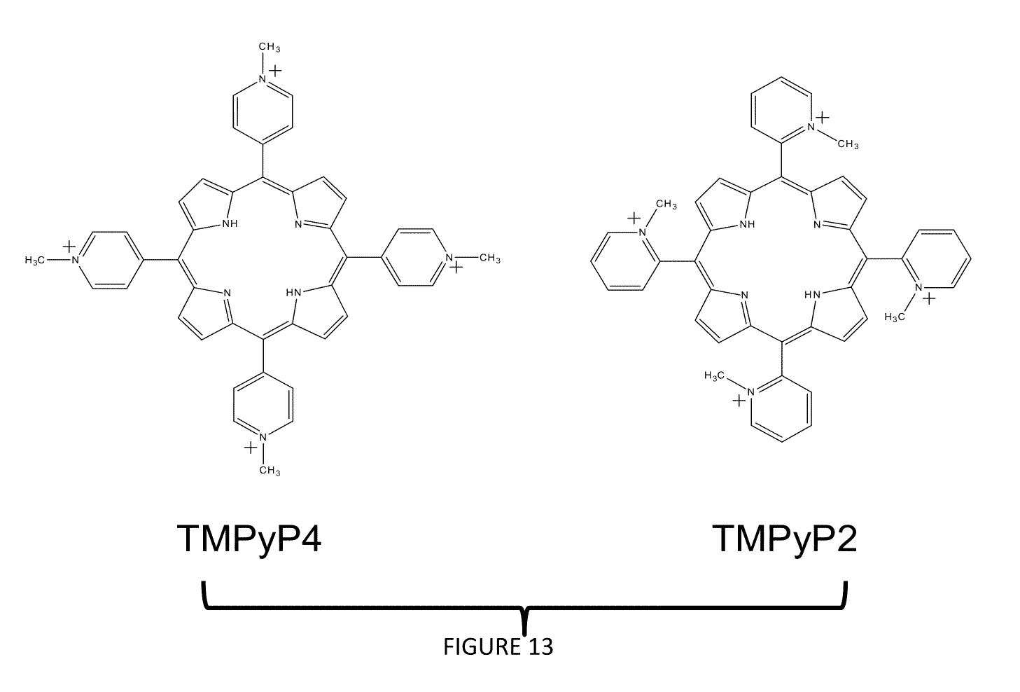

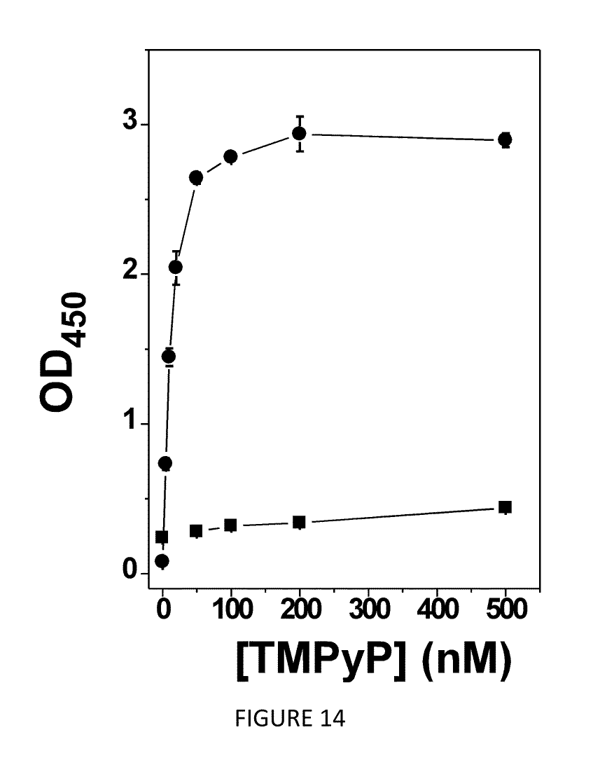

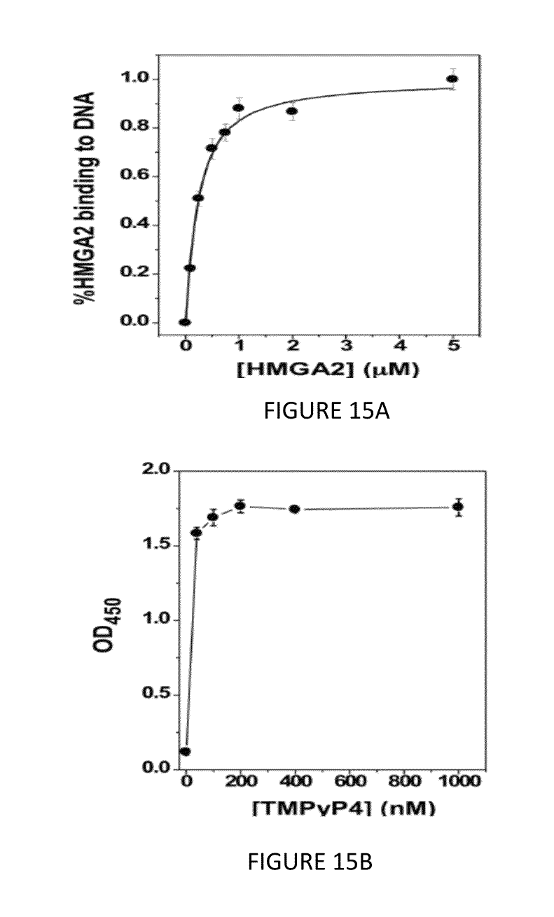

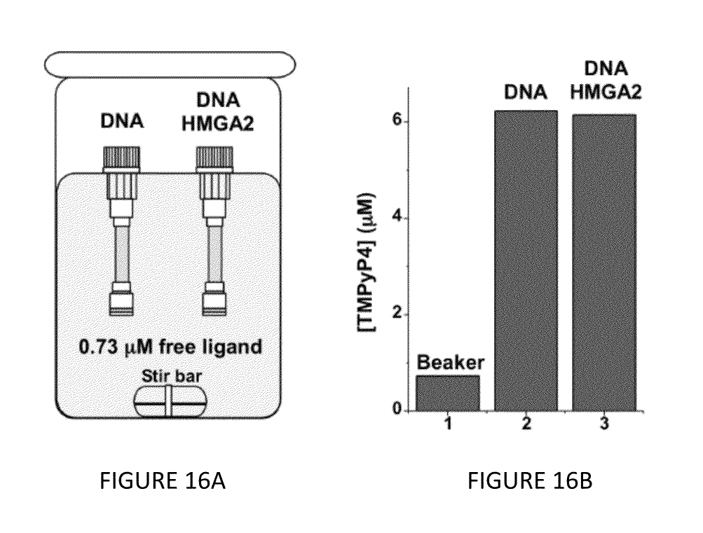

It was also observed that meso-tetra(N-methyl-4-pyridyl) porphine tetratosylate (TMPyP4) (FIG. 13) is able to dramatically enhance the binding of HMGA2 to the target DNA sequence (FL814). To demonstrate the enhancement, a dilution of 1:2500 was used for the second antibody, HRP-linked Anti-rabbit IgG. The incubation time of the second antibody was also shortened from 15 minutes to 5 minutes. In this way, the OD450 value of HMGA2 alone was sufficiently low (0.18). Adding TMPyP4 to the wells dramatically increased the absorbance at OD450 (FIG. 14). In contrast, meso-tetra(N-methyl-2-pyridyl) porphine tetrachloride (TMPyP2), a structurally similar compound, was not able to stimulate the binding of HMGA2. In the presence of TMPyP4, the dissociation constant of HMGA2 to FL814 (K_{d}) was determined to be 0.19±0.02 μM (FIG. 15 (A)), ˜4-fold enhancement of the binding of HMGA2 to FL814. Since FL814 is a hairpin, it is possible that TMPyP4 bound to the loop region of FL814 (FIG. 7 (B) to enhance the binding affinity of HMGA2 to FL814. Nevertheless, using a double stranded DNA oligomer carrying the SELEX1 sequence without the loop region, TMPyP4 was also able to enhance the binding (FIG. 15 (B)), suggesting that the binding of TMPyP4 to the loop region is not the mechanism of the enhancement. In this study, we also carried out a competition dialysis assay (Ren, J. and Chaires, J. B. (1999)) in which a volume of 0.3 ml of FL814 (3 μM) or the HMGA2-FL814 complex (3 μM) was pipeted into a separate 0.3 ml disposable dialyzer and dialyzed against 200 ml of 0.73 μM of TMPyP4 in 2×SSCT buffer extensively. Results in FIGS. 16A-16B show that TMPyP4 was able to bind to both FL814 and the FL814-HMGA2 complex tightly, and HMGA2 did not block the binding of TMPyP4 to FL814. These results also showed that two molecules of TMPyP4 bind to one molecule of FL814. These experiments demonstrate that the methods described in EXAMPLE 5 are valid and can be used to screen activators.

### Example 7

### Netropsin Inhibits the Differentiation of 3T3-L1 Cells to Adipocytes

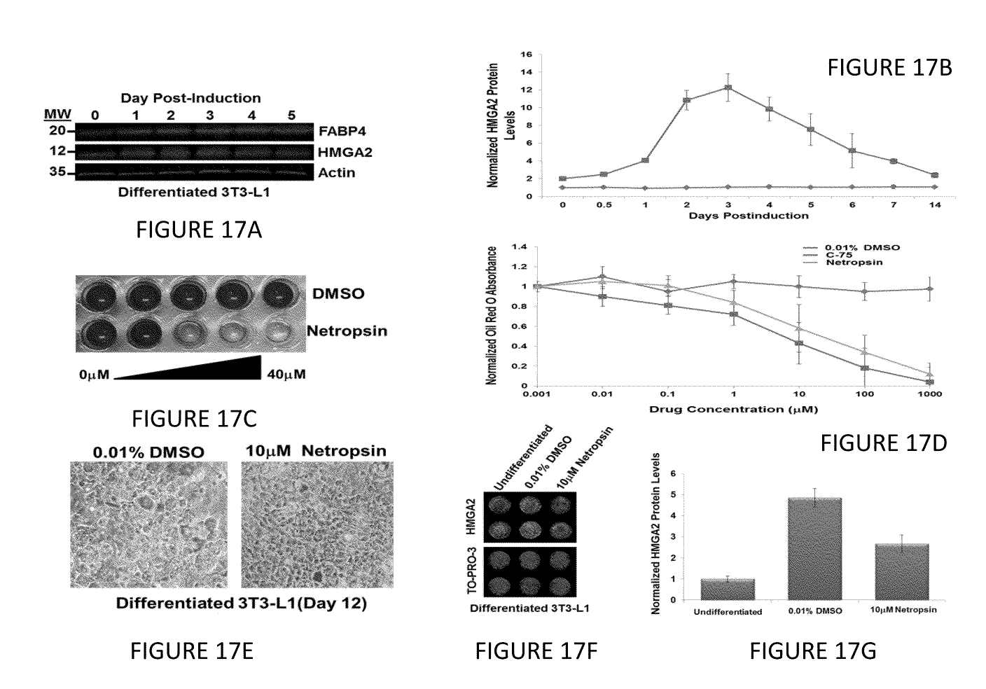

Previous studies showed that HMGA2 plays an important role in the differentiation of the mouse NIH 3T3-L1 preadipocyte cells into adipocytes (Sun, T., et al. (2009)) (FIGS. 17 (A) and (B)). Knockdown of HMGA2 by miRNA let-7 significantly inhibited 3T3-L1 differentiation (Sun, T., et al. (2009)). Since netropsin is a potent inhibitor of HMGA2 binding to AT-rich DNA sequences, this compound should be able to inhibit the differentiation of 3T3-L1 into adipocytes. Indeed, results in FIGS. 17A-17G clearly demonstrate that netropsin strongly inhibited the differentiation of 3T3-L1 cells. At 10 μM, a concentration much lower than its cytotoxicity IC₅₀ of 44.7 μM and above (Zhao, R., et al. (1997)), netropsin was able to inhibit more than 50% of neutral lipid production measured by Oil red O staining in the differentiated cells (FIGS. 17 (D) and (E)). The inhibition IC₅₀ of neutral lipids in the differentiated cells was calculated to be 7.9±1.1 and 3.7±0.8 μM for netropsin and C-75 (an inhibitor of fatty acid synthase), respectively. As expected, HMGA2 was also expressed in fully differentiated 3T3-L1 cells even in the presence of netropsin (FIGS. 17 (F) and (G)). These results strongly suggest that the inhibition of differentiation of 3T3-L1 to adipocytes was likely through a mechanism by which netropsin prevents HMGA2 from binding to its target DNA sequences on the chromosome. It is important to point out here that the direct interactions of netropsin with DNA may also affect other DNA metabolic pathways and therefore inhibit the differentiation. Nevertheless, further studies are needed to determine the inhibition mechanism. Since the over- and/or aberrant-expression of HMGA2 has been directly attributed to the formation of the malignant tumors (Young, A. R. and Narita, M. (2007); Fusco A. and Fedele, M. (2007); Goodwin, G. (1998); Chiappetta, G., et al. (2008)) and the expression level of HMGA2 often correlates with the degree of malignancy, the existence of metastasis, and a poor prognosis (Abe, N., et al. (1999); Meyer, B., et al. (2007)), netropsin and similar compounds should have potent anticancer and anti-metastasis activities by preventing HMGA2 from binding to its target sites on chromosome (Zhao, R., et al. (1997)).

### DISCUSSION

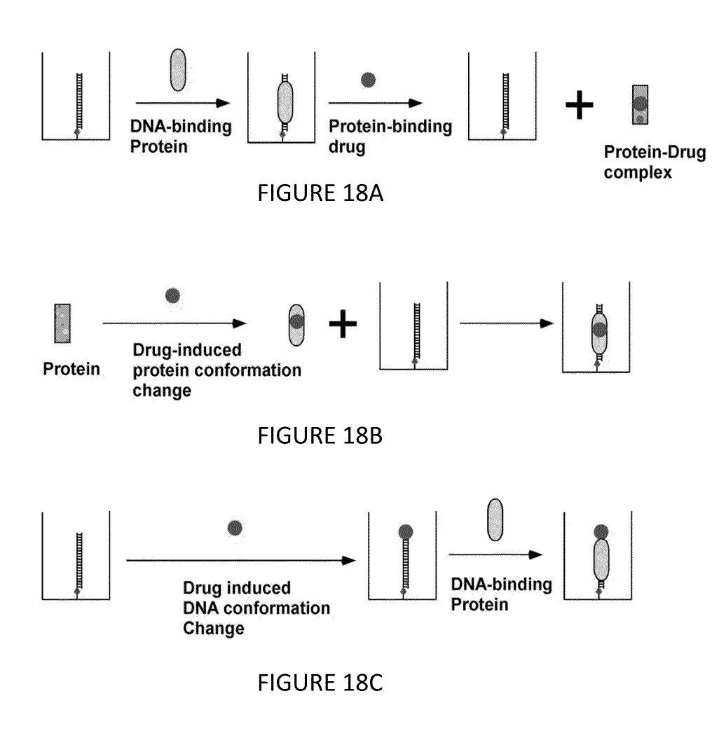

Although the experiments described herein show that PDI-ELISA was successfully used to identify small molecule DNA-binding inhibitors that block the binding of HMGA2 to its DNA-binding sites, the methods described can also be used to screen non-DNA binding ligands that inhibit or activate the binding of a protein to specific DNA sequences. For instance, as described in FIG. 18 (A), PDI-ELISA can be used to screen small molecule compounds that prevent certain transcription factors from binding to the target DNA sequences. The Escherichia coli lac repressor is such a transcription factor that will dissociate from its binding sites, i.e. lac operators, upon binding to an inducer, such as IPTG (Lewis, M., et al. (1996)). One is able to use PDI-ELISA to identify inducers or small molecule ligands to inhibit the binding of transcription factors similar to the lac repressor to their DNA-binding sites. Additionally, certain transcription factors, such as eukaryotic nuclear receptors (Helsen, C., et al. (2012)), do not tightly bind to the target DNA sequences in the absence of a ligand. PDI-ELISA could be a great tool to screen and identify small molecule ligands that enable the transcription factor binding to the target DNA sequence (Underwood, K. F., et al. (2013)) (FIG. 18 (B)). Furthermore, small molecule or protein activators may induce DNA conformational change and therefore greatly enhance the DNA binding of a DNA-binding protein to the target DNA sequence. The methods and kits described herein can be used to identify such activators. Indeed, using PDI-ELISA, it was observed that TMPyP4 significantly enhanced the binding of HMGA2 to the target DNA sequence, i.e. SELEX1 (FIG. 14). This screening method can also be utilized to screen proteins from cellular extracts that inhibit or enhance the targeted protein-nucleic acids interactions. It should be pointed out that the same concept could be used to screen compounds targeting RNA-protein interactions as well. The versatile methods and kits described herein can be used extensively to identify compounds or proteins of interest targeting many specific protein-nucleic acids interactions. In summary, the present invention provides a rapid and sensitive high-throughput screening methods, i.e. PDI-ELISA, and related kits for the identification of inhibitors and activators targeting protein-DNA interactions. Using these methods and kits, several potent inhibitors were identified, including netropsin for HMGA2-DNA interactions from a collection of 29 different DNA-binding compounds. Furthermore, the methods described herein were utilized to determine that TMPyP4 significantly enhances the binding of HMGA2 to the target DNA sequence. Also, the methods described herein were utilized to show that netropsin inhibited the differentiation of the preadipocyte NIH 3T3-L1 cells to adipocytes, most likely through the inhibition of HMGA2 binding to the target DNA sequences.

All patents, patent applications, provisional applications, and publications referred to or cited herein are incorporated by reference in their entirety, including all figures and tables, to the extent they are not inconsistent with the explicit teachings of this specification.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed.

### REFERENCES

Hibma, M. H., Ely, S. J., & Crawford, L. (1994) A non-radioactive assay for the detection and quantitation of a DNA binding protein. Nucleic Acids Res 22, 3806-3807.
Jagelska, E., Brazda, V., Pospisilova, S., Vojtesek, B., & Palecek, E. (2002) New ELISA technique for analysis of p53 protein/DNA binding properties. J. Immunol. Methods 267, 227-235.
Renard, P., Ernest, I., Houbion, A., Art, M., Le, C. H., Raes, M., & Remade, J. (2001) Development of a sensitive multi-well colorimetric assay for active NFkappaB. Nucleic Acids Res. 29, E21.
Kirchler, T., Briesemeister, S., Singer, M., Schutze, K., Keinath, M., Kohlbacher, O., Vicente-Carbajosa, J., Teige, M., Harter, K., & Chaban, C. (2010) The role of phosphorylatable serine residues in the DNA-binding domain of Arabidopsis bZIP transcription factors. Eur. J. Cell Biol. 89, 175-183.
Alonso, R., Onate-Sanchez, L., Weltmeier, F., Ehlert, A., Diaz, I., Dietrich, K., Vicente-Carbajosa, J., & Droge-Laser, W. (2009) A pivotal role of the basic leucine zipper transcription factor bZIP53 in the regulation of Arabidopsis seed maturation gene expression based on heterodimerization and protein complex formation. Plant Cell 21, 1747-1761.
Rosenau, C., Emery, D., Kaboord, B., & Qoronfleh, M. W. (2004) Development of a high-throughput plate-based chemiluminescent transcription factor assay. J. Biomol. Screen. 9, 334-342.
Brand, L. H., Henneges, C., Schussler, A., Kolukisaoglu, H. U., Koch, G., Wallmeroth, N., Hecker, A., Thurow, K., Zell, A., Harter, K. et al. (2013) Screening for Protein-DNA Interactions by Automatable DNA-Protein Interaction ELISA. PLoS. One. 8, e75177.
Young, A. R. & Narita, M. (2007) Oncogenic HMGA2: short or small? Genes Dev. 21, 1005-1009.
Morishita, A., Zaidi, M. R., Mitoro, A., Sankarasharma, D., Szabolcs, M., Okada, Y., D'Armiento, J., & Chada, K. (2013) HMGA2 is a driver of tumor metastasis. Cancer Res. 73, 4289-4299.
Zhou, X., Benson, K. F., Ashar, H. R., & Chada, K. (1995) Mutation responsible for the mouse pygmy phenotype in the developmentally regulated factor HMGI-C. Nature 376, 771-774.
Anand, A. & Chada, K. (2000) In vivo modulation of Hmgic reduces obesity. Nat. Genet. 24, 377-380.
Weedon, M. N., Lettre, G., Freathy, R. M., Lindgren, C. M., Voight, B. F., Perry, J. R., Elliott, K. S., Hackett, R., Guiducci, C., Shields, B. et al. (2007) A common variant of HMGA2 is associated with adult and childhood height in the general population. Nat. Genet. 39, 1245-1250.
Horikoshi, M., Yaghootkar, H., Mook-Kanamori, D. O., Sovio, U., Taal, H. R., Hennig, B. J., Bradfield, J. P., St, P. B., Evans, D. M., Charoen, P. et al. (2013) New loci associated with birth weight identify genetic links between intrauterine growth and adult height and metabolism. Nat. Genet. 45, 76-82.
Copley, M. R., Babovic, S., Benz, C., Knapp, D. J., Beer, P. A., Kent, D. G., Wohrer, S., Treloar, D. Q., Day, C., Rowe, K. et al. (2013) The Lin28b-let-7-Hmga2 axis determines the higher self-renewal potential of fetal haematopoietic stem cells. Nat. Cell Biol. 15, 916-925.
Stein, J. L., Medland, S. E., Vasquez, A. A., Hibar, D. P., Senstad, R. E., Winkler, A. M., Toro, R., Appel, K., Bartecek, R., Bergmann, O. et al. (2012) Identification of common variants associated with human hippocampal and intracranial volumes. Nat. Genet. 44, 552-561.
Cui, T. & Leng, F. (2007) Specific recognition of AT-rich DNA sequences by the mammalian high mobility group protein AT-hook 2: a SELEX study. Biochemistry 46, 13059-13066.
Miao, Y., Cui, T., Leng, F., & Wilson, W. D. (2008) Inhibition of high-mobility-group A2 protein binding to DNA by netropsin: a biosensor-surface plasmon resonance assay. Anal. Biochem. 374, 7-15.
Sun, T., Fu, M., Bookout, A. L., Kliewer, S. A., & Mangelsdorf, D. J. (2009) MicroRNA let-7 regulates 3T3-L1 adipogenesis. Mol. Endocrinol. 23, 925-931.
Zhao, R., al-Said, N. H., Sternbach, D. L., & Lown, J. W. (1997) Camptothecin and minor-groove binder hybrid molecules: synthesis, inhibition of topoisomerase I, and anticancer cytotoxicity in vitro. J. Med. Chem. 40, 216-225.
Erzberger, J. P. and Berger, J. M. (2006) Evolutionary relationships and structural mechanisms of AAA+ proteins. Annu. Rev. Biophys. Biomol. Struct., 35, 93-114.
Stillman, B. (2005) Origin recognition and the chromosome cycle. FEBS Lett., 579, 877-884.
O'Donnell, M., Langston, L. and Stillman, B. (2013) Principles and concepts of DNA replication in bacteria, archaea, and eukarya. Cold Spring Harb. Perspect. Biol., 5, a010108.
Lobe, C. G. (1992) Transcription factors and mammalian development. Curr. Top. Dev. Biol., 27, 351-383.
Osborne, C. K., Schiff, R., Fuqua, S. A. and Shou, J. (2001) Estrogen receptor: current understanding of its activation and modulation. Clin. Cancer Res., 7, 4338s-4342s.
Benizri, E., Ginouves, A. and Berra, E. (2008) The magic of the hypoxia-signaling cascade. Cell Mol. Life Sci., 65, 1133-1149.
Darnell, J. E. Jr (2002) Transcription factors as targets for cancer therapy. Nat. Rev. Cancer, 2, 740-749.
Libermann, T. A. and Zerbini, L. F. (2006) Targeting transcription factors for cancer gene therapy. Curr. Gene Ther., 6, 17-33.
Frank, D. A. (2013) Transcription factor STAT3 as a prognostic marker and therapeutic target in cancer. J. Clin. Oncol., 31, 4560-4561.
Yan, C. and Higgins, P. J. (2013) Drugging the undruggable: transcription therapy for cancer. Biochim. Biophys. Acta, 1835, 76-85.
Heeres, J. T. and Hergenrother, P. J. (2011) High-throughput screening for modulators of protein-protein interactions: use of photonic crystal biosensors and complementary technologies. Chem. Soc. Rev., 40, 4398-4410.
Makley, L. N. and Gestwicki, J. E. (2013) Expanding the number of ‘druggable’ targets: non-enzymes and protein-protein interactions. Chem. Biol. Drug Des., 81, 22-32.
Cui, T., Joynt, S., Morillo, V., Baez, M., Hua, Z., Wang, X. and Leng, F. (2007) Large scale preparation of the mammalian high mobility group protein A2 for biophysical studies. Protein Pept. Lett., 14, 87-91.
Miao, Y., Cui, T., Leng, F. and Wilson, W. D. (2008) Inhibition of high-mobility-group A2 protein binding to DNA by netropsin: a biosensor-surface plasmon resonance assay. Anal. Biochem., 374, 7-15.
Ren, J. and Chaires, J. B. (1999) Sequence and structural selectivity of nucleic acid binding ligands. Biochemistry, 38, 16067-16075.
Zebisch, K., Voigt, V., Wabitsch, M. and Brandsch, M. (2012) Protocol for effective differentiation of 3T3-L1 cells to adipocytes. Anal. Biochem., 425, 88-90.
Chambers, J. W., Howard, S. and LoGrasso, P. V. (2013) Blocking c-Jun N-terminal kinase (JNK) translocation to the mitochondria prevents 6-hydroxydopamine-induced toxicity in vitro and in vivo. J. Biol. Chem., 288, 1079-1087.
Egorina, E. M., Sovershaev, M. A. and Osterud, B. (2006) In-cell Western assay: a new approach to visualize tissue factor in human monocytes. J. Thromb. Haemost., 4, 614-620.
Hibma, M. H., Ely, S. J. and Crawford, L. (1994) A non-radioactive assay for the detection and quantitation of a DNA binding protein. Nucleic Acids Res., 22, 3806-3807.
Rosenau, C., Emery, D., Kaboord, B. and Qoronfleh, M. W. (2004) Development of a high-throughput plate-based chemiluminescent transcription factor assay. J. Biomol. Screen., 9, 334-342.
Brand, L. H., Henneges, C., Schussler, A., Kolukisaoglu, H. U., Koch, G., Wallmeroth, N., Hecker, A., Thurow, K., Zell, A., Harter, K. et al. (2013) Screening for protein-DNA interactions by automatable DNA-protein interaction ELISA. PLoS One., 8, e75177.
Young, A. R. and Narita, M. (2007) Oncogenic HMGA2: short or small? Genes Dev., 21, 1005-1009.
Morishita, A., Zaidi, M. R., Mitoro, A., Sankarasharma, D., Szabolcs, M., Okada, Y., D'Armiento, J. and Chada, K. (2013) HMGA2 is a driver of tumor metastasis. Cancer Res., 73, 4289-4299.
Zhou, X., Benson, K. F., Ashar, H. R. and Chada, K. (1995) Mutation responsible for the mouse pygmy phenotype in the developmentally regulated factor HMGI-C. Nature, 376, 771-774.
Anand, A. and Chada, K. (2000) In vivo modulation of Hmgic reduces obesity. Nat. Genet., 24, 377-380.
Weedon, M. N., Lettre, G., Freathy, R. M., Lindgren, C. M., Voight, B. F., Perry, J. R., Elliott, K. S., Hackett, R., Guiducci, C., Shields, B. et al. (2007) A common variant of HMGA2 is associated with adult and childhood height in the general population. Nat. Genet., 39, 1245-1250.
Horikoshi, M., Yaghootkar, H., Mook-Kanamori, D. O., Sovio, U., Taal, H. R., Hennig, B. J., Bradfield, J. P., St, P. B., Evans, D. M., Charoen, P. et al. (2013) New loci associated with birth weight identify genetic links between intrauterine growth and adult height and metabolism. Nat. Genet., 45, 76-82.
Copley, M. R., Babovic, S., Benz, C., Knapp, D. J., Beer, P. A., Kent, D. G., Wohrer, S., Treloar, D. Q., Day, C., Rowe, K. et al. (2013) The Lin28b-let-7-Hmga2 axis determines the higher self-renewal potential of fetal haematopoietic stem cells. Nat. Cell Biol., 15, 916-925.
Stein, J. L., Medland, S. E., Vasquez, A. A., Hibar, D. P., Senstad, R. E., Winkler, A. M., Toro, R., Appel, K., Bartecek, R., Bergmann, O. et al. (2012) Identification of common variants associated with human hippocampal and intracranial volumes. Nat. Genet., 44, 552-561.
Fusco, A. and Fedele, M. (2007) Roles of HMGA proteins in cancer. Nat. Rev. Cancer, 7, 899-910.
Cui, T. and Leng, F. (2007) Specific recognition of AT-rich DNA sequences by the mammalian high mobility group protein AT-hook 2: a SELEX study. Biochemistry, 46, 13059-13066.
Sun, T., Fu, M., Bookout, A. L., Kliewer, S. A. and Mangelsdorf, D. J. (2009) MicroRNA let-7 regulates 3T3-L1 adipogenesis. Mol. Endocrinol., 23, 925-931.
Zhao, R., al-Said, N. H., Sternbach, D. L. and Lown, J. W. (1997) Camptothecin and minor-groove binder hybrid molecules: synthesis, inhibition of topoisomerase I, and anticancer cytotoxicity in vitro. J. Med. Chem., 40, 216-225.
Goodwin, G. (1998) The high mobility group protein, HMGI-C. Int. J. Biochem. Cell Biol., 30, 761-766.
Chiappetta, G., Ferraro, A., Vuttariello, E., Monaco, M., Galdiero, F., De, S. V., Califano, D., Pallante, P., Botti, G., Pezzullo, L. et al. (2008) HMGA2 mRNA expression correlates with the malignant phenotype in human thyroid neoplasias. Eur. J. Cancer, 44, 1015-1021.
Abe, N., Watanabe, T., Sugiyama, M., Uchimura, H., Chiappetta, G., Fusco, A. and Atomi, Y. (1999) Determination of high mobility group I(Y) expression level in colorectal neoplasias: a potential diagnostic marker. Cancer Res., 59, 1169-1174.
Meyer, B., Loeschke, S., Schultze, A., Weigel, T., Sandkamp, M., Goldmann, T., Vollmer, E. and Bullerdiek, J. (2007) HMGA2 overexpression in non-small cell lung cancer. Mol. Carcinog, 46, 503-511.
Lewis, M., Chang, G., Horton, N. C., Kercher, M. A., Pace, H. C., Schumacher, M. A., Brennan, R. G. and Lu, P. (1996) Crystal structure of the lactose operon repressor and its complexes with DNA and inducer. Science, 271, 1247-1254.
Helsen, C., Kerkhofs, S., Clinckemalie, L., Spans, L., Laurent, M., Boonen, S., Vanderschueren, D. and Claessens, F. (2012) Structural basis for nuclear hormone receptor DNA binding. Mol. Cell Endocrinol., 348, 411-417.
Underwood, K. F., Mochin, M. T., Brusgard, J. L., Choe, M., Gnatt, A. and Passaniti, A. (2013) A quantitative assay to study protein:DNA interactions, discover transcriptional regulators of gene expression, and identify novel anti-tumor agents. J. Vis. Exp., 78, e50512.

We claim:

1. A method of screening agents that inhibit or, alternatively, enable or enhance the sequence-specific binding of a polynucleotide binding protein, the method comprising:
providing a substrate with a plurality of a polynucleotide fragment bound to its surface;
contacting the substrate with an amount of a candidate agent;
contacting the substrate with a plurality of a sequence-specific polynucleotide binding protein, or fragment thereof, wherein the sequence-specific polynucleotide binding protein, or fragment thereof, binds at least a portion of the polynucleotide fragment bound to the surface; and
detecting whether the sequence-specific polynucleotide binding protein, or fragment thereof, is bound to the polynucleotide fragment;
wherein a lack of detection of the sequence-specific polynucleotide binding protein indicates the candidate compound is a compound that inhibits the sequence-specific binding of the polynucleotide binding protein, and
wherein detection of the sequence-specific polynucleotide binding protein indicates the candidate compound is a compound that enhances or enables the sequence-specific binding of the polynucleotide binding protein, or fragment thereof.

2. The method of claim 1, wherein the detecting further comprises:
contacting the substrate with a first antibody specific for the sequence-specific polynucleotide binding protein, or fragment thereof;
contacting the substrate with a detector antibody specific for the first antibody to effect an immunoreaction, the detector antibody comprising a label that provides a detectable signal;
detecting the detectable signal; and
quantifying the detectable signal to determine the amount of the sequence-specific polynucleotide binding protein, or fragment thereof, bound to the polynucleotide fragment;
wherein reduced detection of the detectable signal relative to a control in the absence of the candidate agent indicates the candidate agent inhibits the sequence-specific binding of the polynucleotide binding protein, or fragment thereof; and
wherein increased detection of the detectable signal relative to the control in the absence of the candidate agent indicates the candidate agent enhances or enables the sequence-specific binding of the polynucleotide binding protein, or fragment thereof.

3. The method of claim 2, wherein reduced detection of the detectable signal relative to a control in the absence of the candidate agent indicates the candidate agent is an agent that binds to the polynucleotide fragment and inhibits the binding of the polynucleotide binding protein, or fragment thereof, thereto.

4. The method of claim 2, wherein the candidate agent and the sequence-specific polynucleotide binding protein, or fragment thereof, are contacted together before contacting the substrate.

5. The method of claim 4, wherein reduced detection of the detectable signal relative to a control in the absence of the candidate agent indicates the candidate agent binds to the polynucleotide binding protein, or fragment thereof, and disrupts the sequence-specific binding of the polynucleotide binding protein to the polynucleotide fragment.

6. The method of claim 5, wherein the candidate agent causes a protein conformation change that disrupts the sequence-specific binding of the polynucleotide binding protein, or fragment thereof, to the polynucleotide fragment.

7. The method of claim 1, wherein the polynucleotide fragment is selected from the group consisting of a DNA oligomer, an RNA oligomer, double stranded DNA, double stranded RNA, single stranded DNA, and single stranded RNA.

8. A method of screening for agents that enable the sequence-specific binding of a protein, or fragment thereof, to a polynucleotide fragment, the method comprising:
providing a substrate with a plurality of a polynucleotide fragment bound to its surface;
contacting the substrate with an amount of a candidate agent;
contacting the substrate with a protein, or fragment thereof, that lacks a binding affinity for the polynucleotide fragment; and
detecting whether the protein is bound to the polynucleotide fragment;
wherein detection of the protein signifies the candidate agent enables the sequence-specific binding of the protein, or fragment thereof.

9. The method of claim 8, wherein the detecting further comprises:
contacting the substrate with a first antibody specific for the protein, or fragment thereof;
contacting the substrate with a detector antibody specific for the first antibody to effect an immunoreaction, the detector antibody comprising a label that provides a detectable signal;
detecting the detectable signal; and
quantifying the detectable signal to determine the amount of the protein bound to the polynucleotide fragment;
wherein increased detection of the detectable signal relative to a control in the absence of the candidate agent indicates the candidate agent enables the sequence-specific binding of the protein, or fragment thereof.

10. The method of claim 8, wherein the polynucleotide fragment is selected from the group consisting of a DNA oligomer, an RNA oligomer, double stranded DNA, double stranded RNA, single stranded DNA, and single stranded RNA.

11. The method of claim 9, wherein the candidate agent and the protein, or fragment thereof, are contacted together before contacting the substrate.

12. The method of claim 11, wherein increased detection of the detectable signal relative to a control in the absence of the candidate agent indicates the candidate agent binds to the protein, or fragment thereof, and enables the sequence-specific binding of the protein to the polynucleotide fragment.

13. The method of claim 12, wherein the candidate agent causes a protein conformation change that enables the sequence-specific binding of the protein, or fragment thereof, to the polynucleotide fragment.

14. An enzyme linked immunosorbent assay (ELISA) kit for screening for agents that inhibit or, alternatively enable, the sequence-specific binding of a polynucleotide binding protein, or fragment thereof, the kit comprising:
a substrate for binding a polynucleotide fragment of interest;
a known amount of a polynucleotide binding protein, or fragment thereof;
a known amount of a first antibody specific to the polynucleotide binding protein, or fragment thereof; and
a known amount of a labelled-secondary antibody specific to the first antibody.

15. The kit of claim 14, wherein the polynucleotide fragment of interest is bound to the substrate.

16. The kit of claim 14, wherein the substrate is a multi-well plate.

17. The kit of claim 14, further comprising a blocking agent.

18. The kit of claim 15, wherein the polynucleotide fragment of interest is biotin-labeled and streptavidin is bound to the substrate such that the polynucleotide sequence of interest binds to the substrate via biotin-streptavidin linkage.

19. The kit of claim 15, wherein the polynucleotide fragment of interest is covalently linked to the substrate.

20. The kit of claim 14, wherein the polynucleotide fragment of interest is selected from the group consisting of a DNA oligomer, an RNA oligomer, double stranded DNA, double stranded RNA, single stranded DNA, and single stranded RNA.
